(19) United States
(12) Patent Application Publication
(10) Pub. No.: US 2010/0292175 A1
(43) Pub. Date: Nov. 18, 2010

(54) USE OF HYDROXYFLAVAN DERIVATIVES FOR TASTE MODIFICATION

(75) Inventors: Ludger Wessjohann, Halle (Saale) (DE); Michael Backes, Holzminden (DE); Jakob P. Ley, Holzminden (DE); Susanne Paetz, Hoxter (DE); Katharina Reichelt, Rosenheim (DE)

Correspondence Address: CONNOLLY BOVE LODGE & HUTZ LLP, 1875 EYE STREET, N.W., SUITE 1100, WASHINGTON, DC, 20006, US

(73) Assignees: Leibniz-Institut fur Pflanzenbiochemie, Halle (Saale) (DE); SYMRISE GmbH & Co. KG, Holzminden (DE)

(21) Appl. No.: 12/781,587

(22) Filed: May 17, 2010

Related U.S. Application Data
(60) Provisional application No. 61/178,667, filed on May 15, 2009.

(51) Int. Cl. A61K 31/70; A23L 1/22; A61K 31/35

(52) U.S. Cl. 514/23; 426/533; 514/456

(57) ABSTRACT

The present invention primarily relates to the use of specific compounds of formula (I)

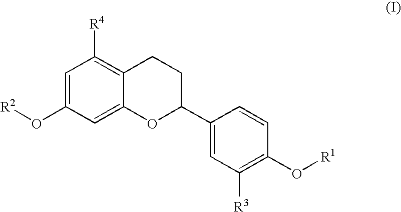

or of specific salts of a compound of formula (I) or mixtures thereof for synergistically enhancing the sweet taste of a sweet-tasting substance.

The present invention primarily relates to the use of compounds of formula (I), to the salts thereof or mixtures thereof for synergistically enhancing the sweet taste of a sweet-tasting substance.

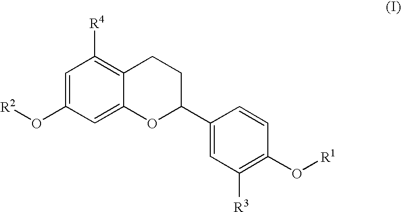

The meaning of the radicals R¹ to R⁴ and the configuration at the chiral carbon atom as well as preferred compounds of formula (I) are described further below.

A further aspect of the present invention relates to the use of compounds of formula (I), to the salts thereof or mixtures thereof for masking or reducing unpleasant taste sensations, in particular for masking or reducing a bitter taste sensation of a bitter-tasting substance.

The present invention further relates to a flavoring composition comprising one or more compounds of formula (I), the salts thereof or mixtures thereof.

Furthermore, the present invention relates to preparations, in particular to preparations used for nutrition, oral care or enjoyment or cosmetic preparations or pharmaceutical preparations intended for oral ingestion, comprising one or more compounds of formula (I), the salts thereof or mixtures thereof.

The present invention also relates to a process for synergistically enhancing the sweet taste of a sweet-tasting substance.

Further aspects of the present invention and preferred embodiments thereof will emerge from the following description and accompanying claims.

Consumers generally have a strong preference for foodstuffs or indulgence foods which have a high sugar content (particularly sucrose=(saccharose), lactose, glucose or fructose or mixtures thereof) due to the sweetness thereof. On the other hand, it is generally known that a high content of readily metabolizable carbohydrates causes a steep rise in blood sugar levels, leads to the formation of fat deposits and ultimately can result in health problems such as being overweight, obesity, insulin resistance, age-onset diabetes and complications thereof. Another particular aggravating factor is that many of the above-mentioned carbohydrates can also have an adverse effect on dental health, as they are decomposed by specific types of bacteria in the oral cavity into lactic acid, for example, and can attack the enamel of milk teeth or adult teeth (caries).

Therefore, it has long been an objective to reduce the sugar content of foodstuffs and/or indulgence foods to the level which is absolutely necessary or below. A suitable measure is to use sweeteners: these are chemically uniform substances which themselves have no, or only a very low calorific value, while at the same time providing a strong sweet taste sensation; in general, the substances are non-cariogenic (a review can be found, for example in Journal of the American Dietetic Association 2004, 104 (2), 255-275).

Although so-called bulk sweeteners such as sorbitol, mannitol or other sugar alcohols are to some extent excellent sweeteners and can also replace to some extent the other foodstuff characteristics of sugars, when ingested too frequently by a proportion of the population they lead to osmotically conditioned digestion problems.

Due to their low application concentration, non-nutritive, high-intensity sweeteners are indeed very suitable for introducing sweetness into foodstuffs, however they often exhibit problems in respect of taste due to dissimilar time-intensity profiles compared to sugar (for example sucralose, steviosides, cyclamate), a bitter and/or astringent aftertaste (for example acesulfame-K, saccharin, steviosides, rebaudiosides) and/or pronounced additional flavor sensations (for example glycerrhyzic acid ammonium salt). Some of the sweeteners are not particularly heat-stable (for example thaumatin, brazzein, monellin), are not stable in every application (for example aspartame) and some have a very long-lasting sweet effect (strong sweet aftertaste, for example saccharin, sucralose).

One possibility—without using non-nutritive sweeteners—is to reduce the sugar content of foodstuffs and/or indulgence foods and to add substances which are sensorially faintly detectable or undetectable and which indirectly or directly enhance the sweetness, as described, for example in WO 2005/041684. However, the substances described in WO 2005/041684 are explicitly of a non-natural origin and thus, from a toxicological point of view, are more difficult to assess than substances of a natural origin, particularly if the latter occur in foodstuffs or indulgence foods or originate from raw materials for the production of foodstuffs or indulgence foods. EP 1 291 342 describes such substances of a natural origin (pyridinium betaines); however, these substances do not influence the sweet taste selectively, but also influence other taste flavors such as umami or saltiness. Furthermore, the disclosed substances can only be purified with great effort.

WO 2007/014879 A1 recommends the use of hesperetin and WO 2007/107596 A1 recommends phloretin to enhance the sweet taste of reduced-sugar preparations used for nutrition or enjoyment. However, an occasional disadvantage when using hesperetin and phloretin is the relatively indistinct sweetness enhancement in foodstuffs and indulgence foods, for example yogurt products which have high contents of proteins, in particular denatured proteins, or polysaccharides. Hesperetin also suffers from the disadvantage that it does not have an adequate effect in very acidic and carbonized applications, such as lemonade or cola drinks.

It is therefore desirable to find substances which, in low concentrations, effectively enhance sweet taste sensations of sweet substances, preferably the sweet taste sensation of reduced-sugar foodstuffs and indulgence foods, in particular of reduced-sugar foodstuffs and indulgence foods which have a low pH value, without having an adverse effect on the rest of the flavor profile.

The primary object of the present invention was to provide substances which are suitable for enhancing, preferably for synergistically enhancing the sweet taste sensation of a sweet tasting substance, which can have wide applications, are preferably easily accessible and preferably occur naturally. In addition, these substances should further preferably be suitable for masking or reducing an unpleasant taste sensation, in particular a bitter taste sensation of a bitter-tasting substance.

In particular, non-nutritive, high-intensity sweeteners often suffer from taste problems (as described above). Although in particular the steviolglycosides which occur naturally in Stevia ssp. or Rubus ssp. (for example stevioside, rebaudioside A-H, dulcosides, rubusosides, suaviosides A, B and g-J) are very effective sweeteners, they have a marked liquorice-like, unpleasant bitter and astringent secondary taste and/or aftertaste in the concentrations required for an adequate sweetening effect (for example 400-600 ppm for rebaudioside A [purity>90%] in soft drinks in order to achieve a sweetness corresponding to a 10% concentration of sucrose).

Particularly in the case of sweet, calorie-free or virtually calorie-free beverages prepared using sweeteners of this type, this unpleasant secondary and/or aftertaste frequently reduces the sensory acceptance and should therefore be masked.

The literature contains a few possibilities for achieving this. Thus, US 2004/0142084 describes alkali metal hydrogen sulfates as masking agents. However, these substances greatly increase the acid content in applications. U.S. Pat. No. 3,924,017 proposes caffeic acid derivatives for masking purposes. A disadvantage is that caffeic acid itself has a slightly bitter taste and slightly suppresses the sweetness so that more sweetener has to be used.

In WO 2006/087991, the unpleasant taste is suppressed using alkamides such as spilanthol. However, the tingling effect of this substance group is often undesirable in this application, thus they cannot be widely used.

An improvement in the taste characteristics, in particular in respect of the aftertaste problem of non-nutritive, high-intensity sweeteners can be achieved using tannic acid, for example as described in WO 98/20753, or phenolic acids, for example as described in U.S. Pat. No. 3,924,017. However, such substances are not particularly stable in applications due to their catechol units and, as typical astringents, can intensify a bitter and/or astringent secondary taste and/or aftertaste.

Not only the above-mentioned stevioglycosides, but also further substances which have a bitter taste or aftertaste and are present in foodstuffs or indulgence foods can severely reduce the quality thereof (for example flavonoid glycosides and limonoids in citrus juices, artificial sweeteners such as aspartame or saccharin, hydrophobic amino acids and/or peptides in cheese), even if these substances are optionally desired in moderation and are characteristic of a foodstuff or indulgence food of this type (for example caffeine in tea or coffee, quinine in so-called bitter lemon beverages, hop extracts in beer).

In particular to reduce the natural content of bitter substances, a subsequent treatment is therefore often necessary, for example by means of extraction as in the decaffeination of tea or coffee, or enzymatically, for example treating orange juice with a glycosidase to destroy the bitter naringin or using special peptidases during the maturing of cheese. This treatment is a strain on the product, generates waste material and also causes, for example, solvent residues and other residues (enzymes) in the products.

It is particularly important to suppress an unpleasant taste sensation, in particular a bitter taste sensation in many pharmaceutical active ingredients, as this can significantly increase the willingness of a patient, especially patients sensitive to bitter substances, for example children, to take the preparation orally. Many pharmaceutical active ingredients, for example aspirin, salicin, paracetamol, ambroxol or quinine, to name just a few as illustrations, have a pronounced bitter, astringent and/or metallic taste or aftertaste.

Although some substances are already known which can suppress (at least partly) the bitter taste, they are often severely restricted in use (cf. Ley JP (2008), Masking Bitter Taste by Molecules, Chemosensory Perception, 1(1), 58-77). Thus, lactisol has been used as a bitter masker, but it simultaneously exhibits a sweetness-masking effect. Sodium salts, for example are also effective bitter maskers, but are naturally salty or contribute to an increase in the sodium concentration in the foodstuff which is undesirable for health reasons.

In connection with the present invention, it was therefore also desirable to find substances which (preferably in small quantities) are able to effectively suppress or at least reduce unpleasant taste sensations, in particular bitter, astringent and/or metallic taste sensations. Such substances should also preferably be widely applicable, easily accessible and naturally occurring.

In view of the preceding explanations, a particularly preferred object of the present invention was to provide substances which are suitable both for synergistically enhancing the sweet taste sensation of a sweet-tasting substance and for masking or reducing a bitter taste sensation of a bitter-tasting substance. Particularly preferably, substances should be provided which can both synergistically enhance the sweet taste sensation of steviolglycosides (for example occurring naturally in Stevia ssp.) and mask or reduce the bitter taste sensation of such steviolglycosides.

A further object of the present invention was to provide a flavoring composition which can synergistically enhance the sweet taste sensation of a sweet-tasting substance and, if the sweet-tasting substance has a bitter secondary taste and/or aftertaste, can preferably also mask or reduce the bitter taste sensation.

Further objects on which the present invention is based will emerge from the following description and the accompanying claims.

The primary object of the present invention is achieved according to the invention using
a compound of formula (I) (i.e. a hydroxyflavan derivative),

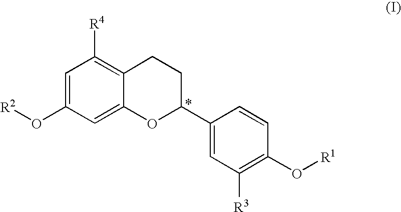

or
a salt of a compound of formula (I)
   or
a mixture of two or more different compounds of formula (I), two or more different salts of compounds of formula (I) or one or more different compounds of formula (I) and one or more different salts of compounds of formula (I),
   wherein the following meanings apply to the groups R¹, R², R³ and R⁴ as well as to the configuration in the compound of formula (I) or independently of one another in each compound of formula (I):
R¹ and R² independently of one another represent hydrogen, methyl or ethyl,
R³ and R⁴ independently of one another represent hydrogen, hydroxyl, methoxy or ethoxy, and
the configuration at the chiral carbon atom (i.e. at the position marked by “*” in the above formula) is (R) or (S),
   for synergistically enhancing the sweet taste of a sweet-tasting substance.

Sweet-tasting substances within the meaning of the present invention are in particular:
sweet-tasting carbohydrates
(for example saccharose, trehalose, lactose, maltose, melizitose, raffinose, palatinose, lactulose, D-fructose, D-glucose, D-galactose, L-rhamnose, D-sorbose, D-mannose, D-tagatose, D-arabinose, L-arabinose, D-ribose, D-gyceraldehyde),
sugar alcohols
(for example erythritol, threitol, arabitol, ribitol, xylitol, sorbitol, mannitol, dulcitol, lactitol),
proteins
(for example miraculin, pentaidin, monellin, thaumatin, curculin, brazzein),
sweeteners
(for example MAGAP, sodium cyclamate, acesulfame K, neohesperidin dihydrochalcone, naringin dihydrochalcone, saccharin, saccharin-sodium salt, aspartame, superaspartame, neotame, alitame, sucralose, lugduname, carrelame, sucrononate, sucrooctate or
naturally occurring sweeteners such as miraculin, curculin, monellin, mabinlin, thaumatin, curculin, brazzein, pentadin, D-phenylalanine, D-tryptophan or extracts or fractions obtained from natural sources, containing such amino acids or proteins, neohesperidin dihydrochalcone, steviolgylcosides, steviosides, steviolbioside, rebaudiosides, rebaudioside A, rebaudioside B, rebaudioside C, rebaudioside D, rebaudioside E, rebaudioside F, rebaudioside g, rebaudioside H, dulcosides, rubusosides, suaviosides A, suaviosides B, suaviosides g, suaviosides H, suaviosides I, suaviosides J, baiyunosides 1 baiyunosides 2, phlomisosides 1, phlomisosides 2, phlomisosides 3, phlomisosides 4, abrusosides A, abrusosides B, abrusosides C, abrusosides D, cyclocaryosides A, cyclocaryosides I, oslandin, polypodoside A, strogin 1, strogin, 2 strogin 4, selligueanin A, dihydroquercetin-3-acetate, perillartin, telosmoside A₁₅, periandrin I-V, pterocaryosides, cyclocaryosides, mukuroziosides, trans-anethol, trans-cinnamaldehyde, bryosides, bryonosides, bryonodulcosides, carnosiflosides, scandenosides, gypenosides, trilobtain, phloridzin, dihydroflavanols, hematoxylin, cyanin, chlorogenic acid, albiziasaponin, telosmosides, gaudichaudioside, mogrosides, hernandulcins, monatin, glycyrrhetinic acid and the derivatives and salts thereof and phyllodulcin).

The naturally occurring sweeteners are used in the context of a use according to the invention preferably in the form of extracts or concentrated fractions of these extracts as sweet-tasting substances (or optionally as both sweet and unpleasant/bitter-tasting substances) (cf. in this respect the description further below), wherein in particular Thaumatococcus extracts (Katemfe bush), extracts of Stevia ssp. (in particular Stevia rebaudiana), Swingle extract (Momordica or Siratia grosvenorii, luo-han-guo), extracts of glycerrhyzia ssp. (in particular glycerrhyzia glabra), Rubus ssp. (in particular Rubus suavissimus), citrus extracts, extracts of Lippia dulcis, buddha tea extracts (Hydrangea dulcis or Hydrangea macrophylla) are used.

Of particular advantage and therefore preferable in the context of the present invention is a use as described above, wherein the or one, more or all the compound(s) of formula (I) is/are respectively selected from the group consisting of the following compounds 1 to 16:
4′,7-dihydroxyflavan (compound 1),
3′,7-dihydroxy-4″-methoxyflavan (compound 2),
4′,7-dihydroxy-3″-methoxyflavan (compound 3),
4′-hydroxy-7-methoxyflavan (compound 4),
4′,7-dimethoxy-3′-hydroxyflavan (compound 5),
3′,7-dimethoxy-4′-hydroxyflavan (compound 6),
3′,4′-dihydroxy-7-methoxyflavan (compound 7),
5,7-dimethoxy-4′-hydroxyflavan (compound 8),
4′-hydroxy-3′,5,7-trimethoxyflavan (compound 9),
3′,4′-dihydroxy-5,7-dimethoxyflavan (compound 10),
4′,5-dihydroxy-3′,7-dimethoxyflavan (compound 11),
3′,4′,5,7-tetrahydroxyflavan (compound 12),
3′,4′,5-trihydroxy-7-methoxyflavan (compound 13),
4′,5,7-trihydroxyflavan (compound 14),
3′,5,7-trihydroxy-4′-methoxyflavan (compound 15),
4′,5,7-trihydroxy-3′-methoxyflavan (compound 16),

the configuration at the chiral carbon atom being (R) or (S) independently of one another in each compound 1 to 16.

The structures of compounds 1 to 16 are provided below for illustration purposes:

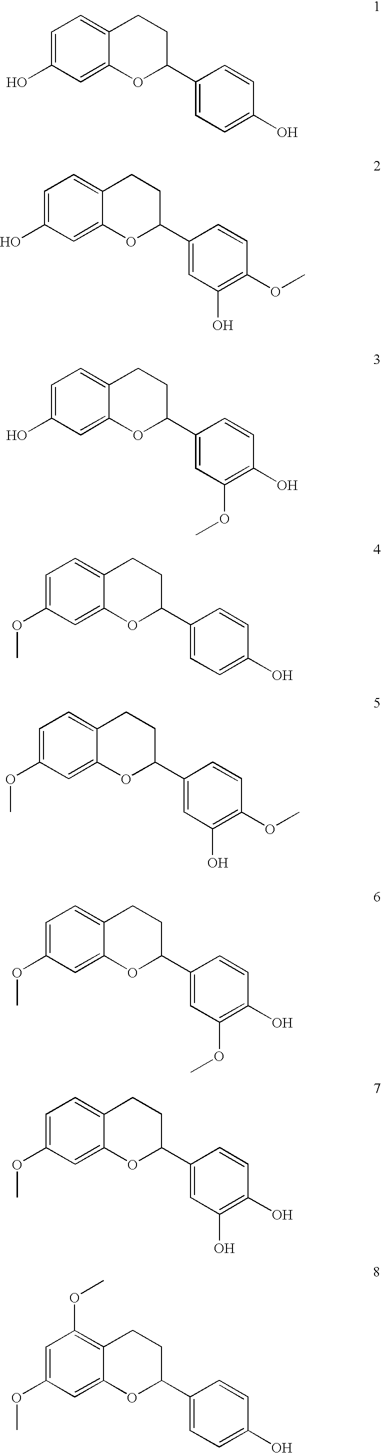

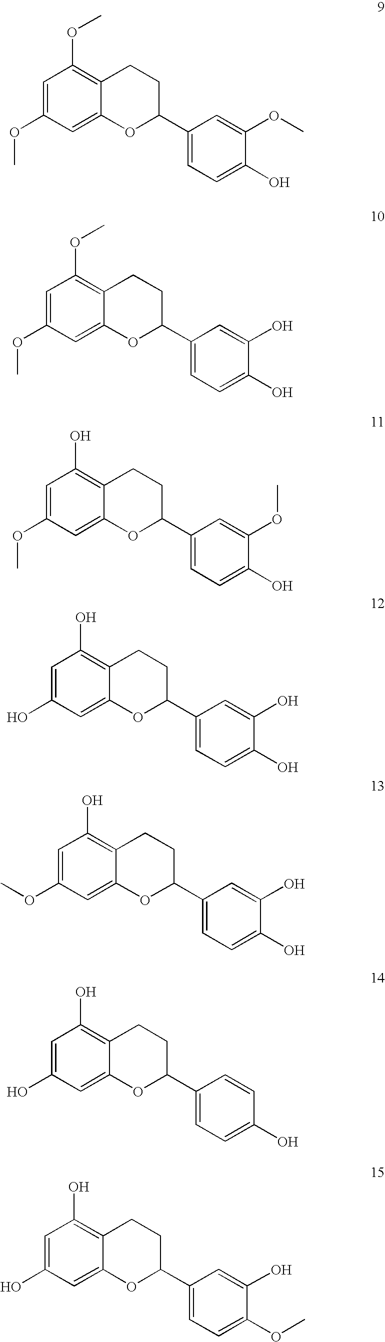

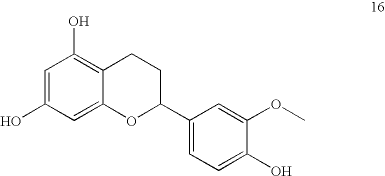

Insofar as compounds of formula (I) which are preferred or particularly preferred within the scope of the present text are mentioned, the salts of such compounds are of course also (particularly) preferably to be used according to the invention. Further details and preferred embodiments of salts to be used according to the invention are described further below.

The synthesis of compounds to be used according to the invention is generally described, for example in J. Agric. Food Chem. 1981, 29, 305-312, in Synth. Commun. 2003, Volume 33, pages 3527-3536 and in Synth. Commun. 1985, Volume 15, pages 1315-1324.

J. Agric. Food Chem. 1981, 29, 305-312 describes, for example in the context of experiments on the structure-effect relationships of specific flavans and flavanones the compounds 2 and 15 to be used according to the invention (denoted there as compounds 28a and 29 a). It is stated therein that they had a primary sweet taste and, at the same time, in relatively high concentrations, a bitter and/or metallic secondary taste. In particular the existence of a 3-hydroxy-4-methoxyphenyl group appeared to be significant within the context of the experiments, but incidentally is not absolutely necessary for the use in the context of the present invention.

This publication does not contain any indication that one of the compounds disclosed therein can synergistically enhance the sweet taste sensation of a sweet-tasting substance. Incidentally, neither is it disclosed that a compound of formula (I) to be used according to the invention can have taste-modulating, in particular masking and/or taste-reducing effects (with respect to an unpleasant taste sensation). See below for aspects in the context of the present invention which relate to the masking or reduction of unpleasant taste sensations.

The compounds of formula (I) to be used according to the invention occur to some extent in various natural origins. The following table shows plants (while naming the respective citation) which apparently contain one or more of the compounds of formula (I) to be used according to the invention. Compounds of formula (I) listed in the following table are to be particularly preferably used in the context of the present invention.

| | Compound(s) of | |
|---|---|---|
| Source/Origin | formula (I) | Citation |
| Bauhinia manca | 1, 3, 4, 6, | Achenbach et al. (1988) |
| | | Phytochemistry, Vol. 27, |
| | | pages 1835-1841 |
| Lycoris aurea | 1, 2, 4 | Yang et al. (2005) Tianran |
| | | Chanwu Yanjiu Yu Kaifa, |
| | | Vol. 17, pages 539-541 |
| Hippeastrum × | 2 | Wink and Lehmann (1996) |
| hortorum | | Botanica Acta, Vol. 109, |
| | | pages 412-421 |
| Brosimum | 1, 2 | Takashima and Ohsaki |
| acutifolium | | (2001) J. Nat. Prod., |
| | | Vol. 64, pages 1493-1496 |
| Knema | 3 | Gonzales et al. (1993) |
| austrosiamensis | | Phytochemistry, Vol. 32, |
| | | pages 433-438 |
| Dracaena | 1, 2, 3 | Masaoud et al. (1995) |
| cinnabaria | | Phytochemistry, Vol. 38, |
| | | pages 745-749 |
| Terminalia | 2, 3 | Garcez et al. (2003) Bio- |
| argenta | | chemical Systematics and |
| | | Ecology, Vol. 31, 229-232 |
| Iranthera | 3 | Filho et al. (1980) Phyto- |
| elliptica | | chemistry, Vol. 19, |
| | | pages 455-459 |
| Iranthera | 3, 8 | Diaz and Diaz (1986) |
| grandis | | Phytochemistry, Vol. 25, |
| | | pages 2395-2398 |
| Zephyranthes | 3 | Saini and ghosal (1984) |
| flava | | Phytochemistry, Vol. 23, |
| | | pages 2415-2422 |
| Mariscus | 3, 5, 9, 10, 11 | Garo et al. (1996) Phyto- |
| psilostachys | | chemistry, Vol. 43, |
| | | pages 1265-1269 |
| Cyclopia | 12 | Kamara et al. (2004) J. |
| subternata | | Agric. Food Chem., Vol. 52, |
| (honeybush) | | pages 5391-5395 |

Accordingly, one or more or all the compounds of formula (I) to be used according to the invention can also be used in the form of plant extracts, in particular in the form of plant extracts from one of the plants mentioned in the above table. The isolation and uses according to the invention of plant extracts is described further below.

Particularly preferably one, more or all compound(s) of formula (I) to be used according to the invention has/have in each case at least one, preferably three, two or one hydroxyl group(s).

A use is further preferred (as described above) wherein for the groups R² and R⁴ in the compound of formula (I) or independently of one another in one, more or all, preferably all, compound(s) of formula (I):
R² and/or R⁴, preferably R² and R⁴, represent hydrogen.

Particularly preferably, for the groups R¹ and R³ in the compound of formula (I) or independently of one another in one, more or all, preferably all, compound(s) of formula (I):
R¹ represents hydrogen or methyl
   and/or
R³ represents hydrogen or hydroxyl.

A use according to the invention (as described above) is most particularly preferred in which for the or one, more or all compounds of formula (I):
R² and R⁴ represent hydrogen,
R¹ represents hydrogen or methyl, and
R³ represents hydrogen or hydroxyl.

A particularly strong synergistic enhancement of the sweet taste of a sweet-tasting substance in the context of the present invention can be achieved according to our own experiments in particular by a use according to the invention (as described above) in which the or one or two compound(s) of formula (I) is/are selected from the group consisting of 4′,7-dihydroxyflavan (compound 1) and 3′,7-dihydroxy-4″-methoxyflavan (compound 2), the configuration at the chiral carbon atom being (R) or (S) independently of one another in each compound 1 or 2.

Compounds 1 and 2 of formula (I), but especially compound 2, are advantageously particularly very suitable for the synergistic enhancement of the sweet taste sensation of a sweet-tasting substance. Furthermore, compounds 1 and 2 which are particularly preferably to be used advantageously have wide applications, are easily accessible and are naturally occurring (as described above). A particularly advantageous, synergistically sweetness-enhancing effect of these compounds, in particular of compound 2, is obtained when the sweet-tasting substance, the sweet taste sensation of which is to be enhanced synergistically according to the invention, is a sugar, in particular sucrose, glucose or fructose or a combination of two or all these sugars. The particularly advantageous synergistically sweetness-enhancing effect of compound 2 is demonstrated further below in exemplary manner based on the enhancement of the sweet taste sensation of sucrose (see Application Example 2).

Surprisingly, compound 2 in particular is also suitable for masking or reducing an unpleasant taste sensation, in particular a bitter taste sensation of a bitter-tasting substance. Details and further aspects relating to the masking or reduction of unpleasant taste sensations are described further below.

Also preferred is the use according to the invention of a mixture of two different compounds of formula (I) (as described above), preferably of two different compounds of formula (I) previously designated as being particularly preferred,

wherein
the groups R¹ to R⁴ of the two compounds are identical and
the configuration at the chiral carbon atom of one compound is (R) and the configuration at the chiral carbon atom of the other compound is (S).

A mixture of this type to be used according to the invention is either a racemic mixture or a non-racemic mixture, but preferably a racemic mixture.

However, a use according to the invention (as described above) is most particularly preferred wherein the or a compound of formula (I) is (S)-3′,7-dihydroxy-4″-methoxyflavan (compound S-2). The structure of compound S-2 is shown below for illustration purposes:

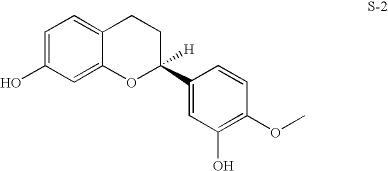

In a further embodiment of the use according to the invention (as described above), it is preferable that in addition to (S)-3′,7-dihydroxy-4″-methoxyflavan (compound S-2), there is no (R)-3′,7-dihydroxy-4″-methoxyflavan or a quantity of (R)-3′,7-dihydroxy-4″-methoxyflavan which is less than the quantity of (S)-3′,7-dihydroxy-4″-methoxyflavan (compound S-2).

For salts, to be used according to the invention, of a compound of formula (I), the information provided above applies accordingly in respect of the preferred meanings of the radicals, but in this case one, more or all hydroxyl groups of the compound of formula (I) (if present) is/are deprotonized. In addition to the (deprotonized) compound(s) of formula (I), a corresponding quantity of counter cations is present, these preferably being selected from the group consisting of: unipositive cations of the first main and secondary groups, ammonium ions, trialkyl ammonium ions, dipositive cations of the second main and secondary groups and tripositive cations of the third main and secondary groups, as well as mixtures thereof. The maximum deprotonization degree of a compound of formula (I) on which a salt of this type is based results from the number of hydroxyl groups of this compound. In turn, there results from the number of deprotonized hydroxyl groups the corresponding amount of counter cations (as a function of their charge). Thus, for example for a compound of formula (I) which has two hydroxyl groups and on which a salt of this type is based, with complete deprotonization of the hydroxyl groups there will be a dinegative anion, from which in turn results the number of positive charges (in this case, two) which has to be provided by the counter cation(s). Particularly preferably, these counter cations are cations which are selected from the group consisting of Na⁺, K⁻, NH₄⁺, Ca²⁺, Mg²⁺, Al³⁺ and Zn²⁺.

Accordingly, particularly preferred is the use according to the invention of a salt or a mixture of
two or more different salts of compounds of formula (I), preferably of compounds of formula (I) designated above as being preferred,
   or
one or more different compounds of formula (I) and one or more different salts of compounds of formula (I),
   as described above,
   wherein the counter cation(s) of the or one, more or all the salts of compounds of formula (I) is/are selected from the group consisting of Na⁺, K⁺, NH₄⁺, Ca²⁺, Mg²⁺, Al³⁺ and Zn²⁺.

The information provided above applies accordingly to the compound(s) of formula (I) on which the salt(s) are based.

In our own experiments in the context of the present invention, it has surprisingly been found that the compounds of formula (I) to be used according to the invention (advantageously already in very low concentrations) can mask or (at least) reduce (in an ideal case completely) the unpleasant taste sensation, in particular the bitter taste sensation of a large number of unpleasant or bitter-tasting substances, in particular of methyl xanthenes, for example caffeine, alkaloids for example quinine, flavonoids for example catechins, naringin, neohesperidin, phenolglycosides for example salicin, arbutin, amygdalin or phenols for example hydroxytyrosol or oleuropein, inorganic salts for example potassium chloride or magnesium sulfate, pharmaceutical active ingredients, for example denatonium benzoate or beta-lactam antibiotics, steviolglycosides for example stevioside or rebaudiosides.

In this respect, it is particularly advantageous that the compounds of formula (I) to be used according to the invention do not have any complexing characteristics. The mentioned advantages apply to an increased extent to compounds 2 which are particularly preferably to be used (as described above). Accordingly, compound 2 is advantageously particularly well suited for both synergistically enhancing a pleasant taste sensation, in particular the sweet taste sensation of a sweet-tasting substance, and for masking or reducing an unpleasant taste sensation, in particular the bitter taste sensation of a bitter-tasting substance.

A further aspect in connection with the present invention therefore relates to the use
of a compound of formula (I)
   or
of a salt of a compound of formula (I)
   or
of a mixture of two or more different compounds of formula (I), two or more different salts of compounds of formula (I) or one or more different compounds of formula (I) and one or more different salts of compounds of formula (I),
   as respectively described above,
   in a mixture comprising
an unpleasant-tasting substance (B)
   or
a both sweet and unpleasant-tasting substance (C),
   (a) for synergistically enhancing the sweet taste of the substance (C)
   and/or
   (b) for masking or reducing the unpleasant taste sensation of the unpleasant-tasting substance (B) or (C).

The information provided above concerning the preferred compounds of formula (I), the salts thereof or mixtures thereof and concerning the sweet-tasting substances applies here accordingly.

A particular object mentioned above of the present invention is achieved by a use according to the invention (as described above) in a mixture comprising
a sweet-tasting substance (A) and an unpleasant-tasting substance (B)
   or
a both sweet and unpleasant-tasting substance (C),
   (a) for synergistically enhancing the sweet taste of the sweet-tasting substance (A) or (C)
   and
   (b) for masking or reducing the unpleasant taste sensation of the unpleasant-tasting substance (B) or (C).

In this respect, the unpleasant taste sensation is particularly preferably a bitter taste sensation and thus the unpleasant-tasting substance (B) or the both sweet and unpleasant-tasting substance (C) is in particular a bitter-tasting substance (B) or a both sweet and bitter-tasting substance (C).

The term “a mixture” in the context of the expression “use ( . . . ) in a mixture” is preferably understood in the scope of the present text as meaning a pharmaceutical preparation intended for oral ingestion, a cosmetic preparation or a preparation used for nutrition, oral care or enjoyment, preferably a preparation according to the invention (as described below).

Sweet-tasting substances (A) are preferably sweet-tasting substances as described above, i.e. in particular sweet-tasting carbohydrates, sugar alcohols, proteins or sweeteners (as respectively described above).

The sweet-tasting substances (A) can have, in addition to the sweet primary taste, one or more further taste sensations (and/or odor sensations), in particular a non-sweet aftertaste sensation. In this respect, the primary taste is understood as meaning the taste sensation which occurs while a substance is in direct contact with the mucous membranes of the oral cavity, in particular with the tongue (usually lasting from a few seconds to minutes). The term “aftertaste” is understood in particular as meaning the taste sensation which persists due to the adhesion of residual amounts of the substance after the oral cavity has been emptied by swallowing and/or spitting out and which can last several minutes to hours.

In particular, as mentioned in the introduction, sweet-tasting substances can have additional unpleasant, in particular bitter taste sensations. Such both sweet and unpleasant-tasting substances are substances (C) which taste both sweet and unpleasant and are preferably to be used in the context of the present invention. The presence of further taste sensations of a substance, but also the intensity of the primary taste itself can vary, for example as a function of the substance concentration, the temperature, the pH value and/or the further substances present in addition to this substance.

Thus for example, when dealing with stevioside or rebaudioside A or another steviolglycoside (as described above), the sweetening power thereof depends on various factors, such as temperature, pH value, concentration and the product to be sweetened. Particularly as a function of the concentration there occurs (with excessive concentrations, in particular with more than 50 ppm, in particular 50 to 2000 ppm, most particularly with 100 ppm to 1000 ppm) a bitter aftertaste which is generally undesirable. In the context of the present invention, stevioside or rebaudioside A are particularly preferred sweet and bitter-tasting substances (C).

The unpleasant or bitter-tasting substances (B) can also have, in addition to an unpleasant taste, further (often not unpleasant) taste and/or odor qualities. Examples of further taste qualities which are not unpleasant within the meaning of the present invention include spicy, umami, sweet, salty, acidic, hot, cooling, warming, burning or tingling sensations.

Unpleasant or bitter-tasting substances with an additional sweet taste sensation are in turn to be allocated to the both sweet and unpleasant-tasting substances (C) within the meaning of the present invention.

Consequently, a both sweet and unpleasant or bitter-tasting substance (C) is preferably a substance selected from the group of sweet-tasting substances, as described above or from the group of unpleasant-tasting substances, as described below.

In the context of the present invention, corresponding unpleasant taste sensations caused by an aftertaste are likewise to be allocated or subordinated to the respective unpleasant taste sensations.

Therefore, unpleasant-tasting substances within the meaning of the present invention are:
substances which have a bitter, astringent, cardboard, chalky, dusty, dry, floury, rancid and/or metallic taste, and
substances which have a corresponding (strongly lasting) aftertaste.

In this respect, a bitter taste sensation often accompanies the astringent, cardboard, chalky, dusty, dry, floury, rancid and/or metallic taste sensations.

Substances which have a bitter, astringent, cardboard, chalky, dusty, dry, floury, rancid or metallic taste are, for example:

xanthine alkaloids, xanthines (caffeine, theobromine, theophylline), alkaloids (quinine, brucine, strychnine, nicotine), phenolic glycosides (for example salicin, arbutin), flavonoid glycosides (for example hesperidine, naringin), chalcones or chalcone glycosides, hydrolysable tannins (gallic—or ellagic acid esters of carbohydrates, for example pentagalloyl glucose), non-hydrolysable tannins (optionally galloylated catechols or epicatechols and oligomers thereof, for example proanthyocyanidines or procyanidines, thearubigin), flavones (for example. quercertin, taxifolin, myricetin), other polyphenols (gamma-oryzanol, coffeic acid or esters thereof), terpenoid bitter substances (for example limonoids such as limonine or nomilin from citrus fruits, lupolones and humolones from hops, iridoids, secoiridoides), absinthin from wormwood, amarogentin from gentian, metal salts (potassium chloride, sodium sulfate, magnesium salts, iron salts, aluminum salts, zinc salts), pharmaceutical active ingredients (for example fluoroquinolone antibiotics, paracetamol, aspirin, beta-lactam antibiotics, ambroxol, propylthiouracil [PROP], guaifenesin), vitamins (for example vitamin H, vitamins from the B group such as vitamin B1, B2, B6, B12, niacin, pantothenic acid), denatonium benzoate or other denatonium salts, sucralose octaacetate, urea, unsaturated fatty acids, in particular unsaturated fatty acids in emulsions, amino acids (for example leucine, isoleucine, valine, tryptophan, proline, histidine, tyrosine, lysine or phenylalanine), peptides (in particular peptides having an amino acid from the group leucine, isoleucine, valine, tryptophan, proline or phenylalanine at the N- or C-terminus).

Substances, in particular flavorings or taste-imparting substances often have a bitter, astringent, cardboard, chalky, dusty, dry, floury, rancid and/or metallic aftertaste, although they have a not unpleasant (primary) taste as defined above (i.e. for example sweet, salty, spicy, sour etc) and/or odor. These flavorings or taste-imparting substances with an unpleasant (after) taste are unpleasant-tasting substances or in particular (if they have a sweet (primary) taste) both sweet and unpleasant-tasting substances within the meaning of the present invention. These flavorings or taste-imparting substances are preferably selected from the group of sweeteners (as described above) or sugar substitutes, i.e. said flavorings or taste-imparting substances preferably have a sweet (primary) taste. Specific examples of such flavorings or taste-imparting substances include aspartame, neotame, superaspartame, saccharin, sucralose, tagatose, monellin, stevioside, rebaudioside, particularly rebaudioside A, mogroside, in particular mogroside V, thaumatin, miraculin, glycyrrhizin, glycyrrhetinic acid or derivatives thereof, cyclamate or the pharmaceutically acceptable salts of the above-mentioned compounds. These flavorings or taste-imparting substances are both sweet and unpleasant/bitter-tasting substances (C) which are preferably to be used for applications according to the invention. The unpleasant taste sensation of these substances, in particular a bitter taste sensation of these substances is masked or reduced in a particularly effective manner in an application according to the invention (as described above).

Further (unpleasant or both sweet and unpleasant-tasting) substances, the unpleasant (secondary) taste of which can advantageously be masked or reduced according to the invention are, for example, flavorings which produce a sweet odor sensation and are preferably selected from the group consisting of vanillin, ethyl vanillin, 2-hydroxy-4-methoxybenzaldehyde, ethylvanillinisobutyrate (=3-ethoxy-4-isobutyryloxybenz-aldehyde), Furaneol® (2,5-dimethyl-4-hydroxy-3(2H)-furanone) and derivatives (e.g. homofuraneol, 2-ethyl-4-hydroxy-5-methyl-3(2H)-furanone), homofuronol (2-ethyl-5-methyl-4-hydroxy-3(2H)-furanone and 5-ethyl-2-methyl-4-hydroxy-3(2H)-furanone), maltol and derivatives (e.g. ethylmaltol), coumarin and derivatives, gamma-lactones (e.g. gamma-undecalactone, gamma-nonalactone), delta-lactones (e.g. 4-methyldeltalactone, massoilactone, deltadecalactone, tuberolactone), methylsorbate, divanillin, 4-hydroxy-2(or 5)-ethyl-5(or 2)-methyl-3(2H)furanone 2-hydroxy-3-methyl-2-cyclopentenones, 3-hydroxy-4,5-dimethyl-2(5H)-furanone, fruit esters and fruit lactones (e.g. acetic acid-n-butylester, acetic acidisoamylester, propionic acid ethylester, butyric acid ethylester, butyric acid-n-butylester, butyric acid isoamylester, 3-methyl-butyric acid ethylester, n-hexanoic acid ethylester, n-hexanoic acid allylester, n-hexanoic acid-n-butylester, n-octanoic acid ethylester, ethyl-3-methyl-3-phenylglycidate, ethyl-2-trans-4-cis-decadienoate), 4-(p-hydroxyphenyl)-2-butanone, 1,1-dimethoxy-2,2,5-trimethyl-4-hexane, 2,6-dimethyl-5-hepten-1-al and phenylacetaldehyde.

Particularly preferred is an application according to the invention (as described above) wherein the bitter-tasting substance (B) or the both sweet and bitter-tasting substance (C), in particular the both sweet and bitter-tasting substance (C) is selected from the group consisting of steviolglycosides, in particular from stevioside and rebaudiosides. The bitter-tasting substance (B) or the both sweet and bitter-tasting substance (C) is preferably selected from the group consisting of rebaudioside A, rubusoside, dulcoside, mogroside, phyllodulcin, glycyrrhetinic acid or extracts of Stevia spp. (in particular Stevia rebaudiana), luo han guo, Rubus suavissimus, Hydrangea dulcis or glycyrrhyza glabra.

The use according to the invention of synergistically sweetness-enhancing compounds of formula (I) or the salts thereof or mixtures thereof can advantageously reduce the total content of sweet-tasting substances (for example in preparations used for nutrition or enjoyment), without reducing the entire sweet taste sensation. This is significant not only for health reasons, but also in respect of the taste characteristics. A sweet (and at high concentrations also bitter) tasting substance, for example stevioside can namely advantageously be used combined with compounds of formula (I) to be used according to the invention or with salts thereof or mixtures thereof (while retaining the sweet taste sensation) in such low concentrations that no, or at least only a reduced bitter (after) taste of the both sweet and bitter-tasting substance, that is to say of stevioside, is detected. Furthermore, the bitter taste sensation (of the both sweet and bitter-tasting substance) can advantageously be masked or at least (further) reduced by a use preferred according to the invention (as described above).

Various studies using Stevia extracts (Yamada A. et al. (1985): Chronic toxicity study of dietary Stevia extracts in F344 rats. In: J. Food Hyg. Soc. Japan. Vol. 26, p. 169-183; Melis, M. S. (1999): Effects of chronic administration of Stevia rebaudiana on fertility in rats. In: J. Ethnopharmacol. Vol. 67, p. 157-161) referred to effects on the male reproductive system, for example reduced spermatogenesis, reduced weight of the sperm vesicles and interstitial cell excrescence in the testicles. It is also known that the leaves of Stevia rebaudiana have been used by Paraguayan Indians in tea as a male contraceptive.

As a result of the synergistically sweetness-enhancing effect of the compounds of formula (I) to be used according to the invention or the salts thereof, in particular due to the synergistically sweetness-enhancing effect of compound 2 (as described above), it is advantageously possible to reduce the amount of Stevia extract or steviolglycosides, in particular stevioside required for a specific degree of sweetness, by combining with one or more compounds of formula (I) or salts thereof (while retaining the desired degree of sweetness), so that disadvantageous effects (which may be present and are described above) can be reduced or avoided.

In addition, a reduction in the required amount of Stevia extract or steviolglycosides can also entail financial savings in addition to the advantages which have already been mentioned.

As already mentioned, one or more or all the compounds of formula (I) to be used according to the invention can also be used in the form of plant extracts.

For this purpose, the plant extracts are preferably obtained from the corresponding fresh or dried plants or parts of plants. The dried plant parts (for example fresh or dried roots, root bark, tubers, bulbs, other subterranean or aerial storage organs, spurious fruits, fruits, seeds, bark, wood, pulp, bast, stalks, stems, leaves or blossom ([parts]) preferably in crushed form, are extracted with a solvent suitable for foodstuffs and indulgence foods at temperatures of 0° C. up to the boiling point of the respective solvent or solvent mixture, then filtered and the filtrate is partly or completely concentrated, preferably by distillation, freeze or spray drying. The raw extract obtained thus can then be further worked up, for example counter-extracted, purified by distributing, absorption, exclusion, affinity or ion chromatography, distilled, sublimated, purified with adsorbents such as active carbon, bentonite, kieselguhr etc, treated enzymatically (for example with glycosidases to increase the yield of non-sugar-containing molecules), with acid (for example under pressure), with suitable basic solutions, for example of hydroxides, carbonates or hydrogen carbonates of sodium, potassium, calcium, magnesium and zinc, with acidic ion exchangers or with water vapor, mixed with an auxiliary and carrier usually at pressures of 0.01 mbar to 100 bar, preferably 1 mbar to 20 bar and optionally dried (for example spray-dried) and/or taken up in a solvent suitable for foodstuffs and indulgence foods.

Solvents suitable for extraction for foodstuffs and indulgence foods include in particular water, ethanol, methanol, propylene glycol, glycerin, acetone, dichloromethane, ethyl acetate, diethyl ester, hexane, heptane, triacetin, vegetable oils or fats, supercritical carbon dioxide and mixtures thereof.

Preferred auxiliaries or carriers include maltodextrin, starch, natural or artificial polysaccharides and/or vegetable gums such as modified starches or gum arabic, coloring agents, for example permitted foodstuff dyes, coloring plant extracts, stabilizers, preservatives, antioxidants, viscosity-influencing substances.

Accordingly, the present invention also relates to the use of a plant extract comprising
a compound of formula (I)
   or
a salt of a compound of formula (I)
   or
a mixture of two or more different compounds of formula (I), two or more different salts of compounds of formula (I) or one or more different compounds of formula (I) and one or more different salts of compounds of formula (I),
   as respectively described above,
   for synergistically enhancing the sweet taste of a sweet-tasting substance.

As already stated, compounds of formula (I) to be used according to the invention are also advantageously suitable for masking or reducing unpleasant, in particular bitter, taste sensations.

Accordingly, the present invention relates in particular to a use according to the invention of a plant extract (as described above) in a mixture comprising
a sweet-tasting substance (A) and an unpleasant, in particular a bitter, tasting substance (B)
   or
a both sweet and unpleasant, in particular bitter, tasting substance (C)
   (a) for synergistically enhancing the sweet taste of the sweet-tasting substance (A) or (C)
   and
   (b) for masking or reducing the unpleasant taste sensation of the unpleasant-tasting substance or of the bitter taste sensation of the bitter-tasting substance (B) or (C).

In this respect, the sweet-tasting substance (A) and the bitter-tasting substance (B) or the both sweet and unpleasant or bitter-tasting substance (C) are in each case not a constituent of the plant extract used.

In the context of applications according to the invention, the compounds of formula (I) to be used according to the invention are advantageously particularly very suitable for use with steviolglycosides.

Accordingly, a use according to the invention described above of a plant extract is also particularly preferred, the bitter-tasting substance (B) or the both sweet and bitter-tasting substance (C) being selected from the group consisting of steviolglycosides, in particular from stevioside and rebaudiosides, being preferably selected from the group consisting of rebaudioside A, rubusoside, dulcoside, mogroside, phyllodulcin, glycyrrhetinic acid or extracts of Stevia spp. (in particular Stevia rebaudiana), luo han guo, Rubus suavissimus, Hydrangea dulcis or glycyrrhyza glabra.

A plant extract to be used according to the invention particularly preferably comprises a compound 2 (as described above). For the rest, the information provided above applies accordingly to the preferred selection of compounds of formula (I). The information provided above also applies accordingly to the meaning of substances (A), (B) and (C).

In a use according to the invention, it can be advantageous if not all unpleasant or bitter-tasting nuances are (completely) masked, since they may possibly even be desired.

A use according to the invention (as described above) is preferably a use in a pharmaceutical preparation intended for oral ingestion, a cosmetic preparation or a preparation used for nutrition, oral care or enjoyment. Preparations according to the invention and the preferred embodiments thereof are described further below.

According to a further aspect of the present invention, a compound of formula (I) to be used according to the invention or a salt of a compound of this type or a mixture thereof (as described above) is preferably used in a flavoring composition for synergistically enhancing the sweet taste of a sweet-tasting substance and/or for masking or reducing a bitter taste sensation of a bitter-tasting substance. A flavoring composition of this type is advantageously particularly very suitable for synergistically enhancing a sweet taste sensation and/or for masking or reducing a bitter taste sensation of a both sweet and bitter-tasting substance.

Accordingly, the present invention also relates to a flavoring composition for synergistically enhancing the sweet taste of a sweet-tasting substance (A) and/or for masking or reducing a bitter taste sensation of a bitter-tasting substance (B), preferably for synergistically enhancing the sweet taste and for masking or reducing the bitter taste sensation of a both sweet and bitter-tasting substance (C), comprising or consisting of

(i)—a compound of formula (I)
or
   a salt of a compound of formula (I)
or
   a mixture of two or more different compounds of formula (I), two or more different salts of compounds of formula (I) or one or more different compounds of formula (I) and one or more different salts of compounds of formula (I),
      as respectively described above,
      and
      (ii) one or more flavoring and/or taste-imparting substances, where it/they is/are not a compound of formula (I) or a salt thereof and is/are preferably selected from the group consisting of flavorings which produce a sweet taste sensation, in particular vanillin, ethyl vanillin, ethylvanillinisobutyrate (=3-ethoxy-4-isobutyryloxybenzaldehyde), Furaneol® (2,5-dimethyl-4-hydroxy-3(2H)-furanone) and derivatives (e.g. homofuraneol, 2-ethyl-4-hydroxy-5-methyl-3(2H)-furanone), homofuronol (2-ethyl-5-methyl-4-hydroxy-3(2H)-furanone and 5-ethyl-2-methyl-4-hydroxy-3(2H)-furanone), maltol and derivatives (e.g. ethylmaltol), coumarin and derivatives, gamma-lactones (e.g. gamma-undecalactone, gamma-nonalactone), delta-lactones (e.g. 4-methyldeltalactone, massoilactone, deltadecalactone, tuberolactone), methylsorbate, divanillin, 4-hydroxy-2(or 5)-ethyl-5(or 2)-methyl-3(2H)furanone 2-hydroxy-3-methyl-2-cyclopentenones, 3-hydroxy-4,5-dimethyl-2(5H)-furanone, fruit esters and fruit lactones (e.g. acetic acid-n-butylester, acetic acid isoamylester, propionic acid ethylester, butyric acid ethylester, butyric acid-n-butylester, butyric acid isoamylester, 3-methyl-butyric acid ethylester, n-hexanoic acid ethylester, n-hexanoic acid allylester, n-hexanoic acid-n-butylester, n-octanoic acid ethylester, ethyl-3-methyl-3-phenylglycidate, ethyl-2-trans-4-cis-decadienoate), 4-(p-hydroxyphenyl)-2-butanone, 1,1-dimethoxy-2,2,5-trimethyl-4-hexane, 2,6-dimethyl-5-hepten-1-al and phenylacetaldehyde,
      and/or
      (iii) one or more (further)
substance(s) for masking or reducing a bitter taste sensation, where it/they is/are not a compound of formula (I) or a salt thereof and is/are preferably selected from the group consisting of sodium salts (for example sodium chloride, sodium lactate, sodium citrate, sodium acetate, sodium gluconoate), homoeriodictyol or the sodium salts thereof, 2,4-dihydroxybenozoic acid vanillylamide, gamma-amino butyric acid, pellitorine (in particular as described in EP 2008530 A1) and gingerdiones
   and/or
substance(s) for enhancing a sweet taste sensation, where it/they is/are not a compound of formula (I) or a salt thereof and is/are preferably selected from the group consisting of hesperetin (in particular as disclosed in WO 2007/014879), hydroxyphenylalkadiones (in particular as described in WO 2007/003527), deoxybenzoins (in particular as described in WO 2006/106023 and in German patent application with file reference 10 2009 002 268.6), 4-hydroxychalcones (in particular as described in WO 2007/107596), propenylphenylglycosides (chavicol glycosides) (in particular as described in EP 1 955 601 A1) and divanillins (in particular as described in WO 2004/078302).

The above-mentioned documents form part of this application by way of reference with respect to the corresponding compounds disclosed therein.

The information provided above applies accordingly to preferably contained compounds of formula (I) or salts or mixtures thereof as well as to the substances (A), (B) and (C).

The present invention also relates to the use of a flavoring composition (as described above) in a mixture comprising
a sweet-tasting substance (A) and/or a bitter-tasting substance (B)
   or
a both sweet and bitter-tasting substance (C)
   (a) for synergistically enhancing the sweet taste of the sweet-tasting substance (A) or (C)
   and/or
   (b) for masking or reducing the unpleasant taste sensation of the unpleasant-tasting substance (B) or (C).

The use of a flavoring composition (as described above) is particularly preferred wherein the bitter-tasting substance (B) or the both sweet and bitter-tasting substance (C) is selected from the group consisting of stevioglycosides, in particular of stevioside and rebaudiosides, is preferably selected from the group consisting of rebaudioside A, rubusoside, dulcoside, mogroside, phyllodulcin, glycyrrhetinic acid or extracts of Stevia spp. (in particular Stevia rebaudiana), luo han guo, Rubus suavissimus, Hydrangea dulcis or glycyrrhyza glabra.

In particular, the present invention also relates to a use of a flavoring composition according to the invention (as described above) for synergistically enhancing the sweet taste of a sweet-tasting substance and/or for masking or reducing a bitter taste sensation of a bitter-tasting substance in a pharmaceutical preparation intended for oral ingestion, a cosmetic preparation or a preparation used for nutrition, oral care or enjoyment, in particular in preparations according to the invention as described below. A flavoring composition according to the invention is particularly preferably used to improve the sensory profile of sweet products which are to be consumed orally.

According to a further aspect of the present invention, the compounds of formula (I) to be used according to the invention or the salts thereof or mixtures thereof or flavoring compositions containing compounds or salts of this type are used in a preparation, in particular in a preparation used for nutrition, oral care or enjoyment or in cosmetic preparations (particularly for application to the head region) or in pharmaceutical preparations intended for oral ingestion. Preparations of this type generally comprise one or more sweet-tasting and/or unpleasant, in particular bitter, tasting substances or both sweet and unpleasant, or that is to say bitter, tasting substances (as respectively described above).

Accordingly, the present invention also relates to a preparation comprising the ingredients:

(I)—a compound of formula (I)
or
   a salt of a compound of formula (I)
or
   a mixture of two or more different compounds of formula (I), two or more different salts of compounds of formula (I) or one or more different compounds of formula (I) and one or more different salts of compounds of formula (I),
      as respectively described above,
      and
      (II) one or more sweet-tasting substances (A) and/or both sweet and bitter-tasting substances (C), the substances(s) (A) or (C) not being a compound of formula (I) or a salt thereof,
      the total amount of compounds of formula (I) or salts of the compound of formula (I) in the preparation being sufficient for synergistically enhancing the sweet taste sensation of the substance(s) (A) or (C).

According to a preferred embodiment of the preparation according to the invention (as described above), said preparation comprises a flavoring composition according to the invention (as described above) which contains the ingredient (I) of the preparation, i.e. one or more compounds of formula (I) and/or salts thereof.

The information provided above applies accordingly in each case to the substances (A) and (C) and to the compounds of formula (I), the salts and mixtures thereof as well as to the preferred selection thereof.

A preparation according to the invention (as described above) is preferred where the preparation is a preparation used for nutrition, oral care or enjoyment or a cosmetic preparation (in particular for application to the head region) or a pharmaceutical preparation intended for oral ingestion.

A preparation according to the invention used for nutrition, oral care or enjoyment or a cosmetic preparation or a pharmaceutical preparation intended for oral ingestion (as described above) is particularly preferred, wherein the preparation contains 0.0001% by weight (1 ppm) to 0.5% by weight (5000 ppm), preferably 0.0001% by weight (1 ppm) to 0.1% by weight (1000 ppm), particularly preferably 0.001% by weight (10 ppm) to 0.05 by weight (500 ppm) of ingredient (I), based on the total weight of the preparation.

Preparations according to the invention can also be present as a semi-finished product or as a seasoning mixture.

A preparation according to the invention (as described above) is particularly preferred wherein the preparation is a semi-finished product suitable for the production of a preparation used for oral care or enjoyment or a cosmetic preparation or a pharmaceutical preparation intended for oral ingestion.

A semi-finished product (as described above) is particularly preferred wherein said semi-finished product contains 0.0001% by weight to 95% by weight, preferably 0.001% by weight to 95% by weight, preferably 0.001% by weight to 80% by weight, particularly preferably 0.01% by weight to 50% by weight of ingredient (I), based on the total weight of the semi-finished product.

A preparation according to the invention (as described above) is particularly preferred wherein the total amount of compounds of formula (I) or salts of the compound of formula (I) in the preparation suffices in imparting the same or an enhanced sweetness sensation when compared to a preparation which, with an otherwise identical composition, does not contain a compound of formula (I) or salt of the compound of formula (I) but does contain at least 1.05 times the amount of sweet-tasting substance(s) (A) or (C).

A preparation according to the invention (as described above) is most particularly preferred wherein ingredient (I) comprises or consists of a compound 1 and/or a compound 2, preferably a compound 2, as respectively described above, in each compound (independently of one another) the configuration at the chiral carbon atom being (R) or (S). A preparation according to the invention is also particularly preferred, comprising as or in ingredient (I) a mixture of two different compounds of formula (I), the groups R¹ to R⁴ of the two compounds being identical and the configuration at the chiral carbon atom being different, i.e. a mixture of two enantiomers (as described above).

Preparations used for nutrition or enjoyment within the meaning of the present invention include, for example bakery products (for example bread, dry biscuits, cakes, other cookies), confectionery (for example chocolate, chocolate bar products, other products in bar form, fruit gums, hard and soft caramels, chewing gum); alcoholic or non-alcoholic beverages (for example coffee, tea, wine, beverages containing wine, beer, beverages containing beer, liqueurs, schnapps, brandies, sodas containing fruit, isotonic beverages, soft drinks, nectars, fruit and vegetable juices, fruit or vegetable preparations); instant beverages (for example instant cocoa beverages, instant tea beverages, instant coffee beverages); meat products (for example ham, fresh or raw sausage preparations, seasoned or marinated fresh or salt meat products); eggs or egg products (dried egg, egg white, egg yolk); cereal products (for example breakfast cereals, muesli bars, precooked finished rice products); milk products (for example milk beverages, ice milk, yogurt, kefir, cream cheese, soft cheese, hard cheese, powdered milk, whey, butter, buttermilk, partially or fully hydrolyzed milk protein-containing products); products from soya protein or other soybean fractions (for example soya milk and products produced therefrom, soya lecithin-containing preparations, fermented products such as tofu or tempeh or products produced therefrom, soy sauces); fruit preparations (for example jams, sorbets, fruit sauces, fruit fillings); vegetable preparations (for example ketchup, sauces, dried vegetables, deep-frozen vegetables, precooked vegetables, vegetables in vinegar, preserved vegetables); snack foods (for example baked or fried potato chips or potato dough products, bread dough products, corn or peanut-based extrudates); fat and oil-based products or emulsions thereof (for example mayonnaise, remoulade, dressings, seasoning preparations); other ready meals and soups (for example powdered soups, instant soups, precooked soups), spices, seasoning mixtures and in particular seasonings which are used, for example, in the snacks sector. The preparations within the meaning of the invention can also be used as semi-finished products for the production of further preparations used for nutrition or enjoyment. The preparations within the meaning of the invention can also be present as capsules, tablets (uncoated and coated tablets, for example with an enteric coating), sugar-coated pills, granules, pellets, solids mixtures, dispersions in liquid phases, as emulsions, powders, solutions, pastes or as other preparations which can be swallowed or chewed as food supplements.

Pharmaceutical preparations intended for oral ingestion within the meaning of the present invention include preparations which are present, for example as capsules, tablets (uncoated and coated tablets, for example with an enteric coating), sugar-coated pills, granules, pellets, solids mixtures, dispersions in liquid phases, as emulsions, powders, solutions, pastes or as other preparations which can be swallowed or chewed and are used as prescription-only medicines, drugstore-only medicines or other medicaments or as food supplements.

Preparations used for oral care within the meaning of the present invention include in particular oral and/or dental care products, such as toothpastes, dental gels, dental powders, mouthwash, chewing gum and other oral care products. Oral care preparations which contain an extract or constituents of an extract of Stevia ssp are particularly preferred. The compounds of formula (I), in particular the compounds of formula (I) designated above as being preferred are advantageously particularly very suitable, in preparations containing steviolglycosides and used for oral care, for masking or reducing a bitter taste sensation of the steviolglycosides, in particular of stevioside and/or rebaudioside A.

Cosmetic preparations, in particular cosmetic preparations for applying to the head region are, within the scope of the present invention, preferably cosmetic preparations which contain at least one unpleasant, in particular a bitter, tasting substance (B) and can come into contact with the oral cavity even when applied correctly to the skin. Preparations of this type are, for example cosmetic preparations to be applied to the head region, such as soaps, other cleansing or care products for the area of the face, face creams, lotions or ointments, sunscreen, beard cleaning or care products, shaving foams, soaps or gels, lipsticks or other lip cosmetics or lip care products.

Ingredient (II) of a preparation according to the invention (as described above) preferably comprises:
one or more both sweet and bitter-tasting substances (C) (as described above)
   and/or
one or more bitter-tasting substances (B), the substance(s) (B) not being a compound of formula (I) or a salt thereof,
   the total amount of compounds of formula (I) or salts of the compound of formula (I) in the preparation being sufficient for masking or reducing the bitter taste sensation of the substance(s) (B) or (C).

The information provided above also applies accordingly to the substances (B) and the preferred selection thereof (as to the substances (A) and (C)).

The present invention relates particularly preferably to a preparation according to the invention (as described above) wherein ingredient (II) comprises one or more both sweet and bitter-tasting substances (C), the total amount of compounds of formula (I) or salts of the compound of formula (I) in the preparation being sufficient for both synergistically enhancing the sweet taste sensation of the both sweet and bitter-tasting substance(s) (C) and for masking or reducing the bitter taste sensation of the both sweet and bitter-tasting substance(s) (C).

A preparation according to the invention (as described above), in particular a preparation used for nutrition, enjoyment or oral care is particularly preferred in which one, more or preferably all the both sweet and bitter-tasting substances (C) (if present) are selected from the group consisting of steviolglycosides, in particular of stevioside and rebaudioside A, preferably selected from the group consisting of rubusoside A, rubusoside, dulcoside, mogroside, phyllodulcin, glycyrrhetinic acid or extracts of Stevia spp. (in particular Stevia rebaudiana), luo han guo, Rubus suavissimus, Hydrangea dulcis or glycyrrhyza glabra.

A preparation according to the invention (as described above) is further preferred in which the total amount of bitter-tasting substances (B) and/or both sweet and bitter-tasting substances (C) in the preparation is sufficient to be detected as a bitter taste in a comparative preparation which, with an otherwise identical composition, does not contain a compound of formula (I) or a salt of the compound of formula (I), and the total amount of compounds of formula (I) or salts of the compound of formula (I) in the preparation is sufficient for masking or, compared with the comparative preparation, for reducing the bitter taste of the substance(s) (B) or (C).

A preparation according to the invention preferably also comprises at least one further substance for masking or reducing a bitter, metallic, chalky, acidic or astringent taste sensation and/or for enhancing a sweet, salty or umami taste sensation. Preferred further substances for masking or reducing an unpleasant taste sensation and/or for enhancing a pleasant taste sensation are described further below.

A preparation according to the invention (as described above) is most particularly preferred wherein
ingredient (I) is present in the form of a plant extract (or a fraction thereof), i.e. in the form of a plant extract comprising a compound of formula (I), a salt of a compound of formula (I) or a mixture thereof (as described above),
   and/or
ingredient (II) is present in the form of a plant extract, preferably an extract of Stevia ssp., in particular an extract of Stevia rabaudiana (or a fraction thereof), i.e. in the form of a plant extract, comprising one or more sweet-tasting substances (A), bitter-tasting substances (B) and/or both sweet and bitter-tasting substances (C) (as respectively described above).

It is particularly advantageous if both ingredient (I) and ingredient (II) are present as a plant extract (or a fraction thereof), ingredient (II) preferably being present in the form of an extract of Stevia rabaudiana. Further details concerning plant extracts comprising one or more compounds of formula (I) or the salts thereof or mixtures thereof as well as the extraction thereof have been described above.

A preparation according to the invention, in particular a preparation or semi-finished product used for nutrition, oral care or enjoyment or a cosmetic preparation or semi-finished product or a pharmaceutical preparation or semi-finished product intended for oral ingestion (as respectively described above) is also preferred which comprises a flavoring composition according to the invention (as described above) which in turn contains ingredient (I) and optionally ingredient (II) of the preparation. The proportion of a flavoring composition according to the invention in such a preparation according to the invention is preferably 0.000001% by weight to 95% by weight, based on the total weight of the preparation.

A preparation according to the invention can also contain conventional active ingredients, basic substances, auxiliaries and additives for preparations used for nutrition, oral care or enjoyment or oral pharmaceutical preparations (i.e. pharmaceutical preparations intended for oral use) or cosmetic preparations (in particular those for application to the head region) in quantities of from 0.9 to 99.999999% by weight, preferably from 10 to 80% by weight, based on the total weight of the preparation. In particular, a preparation according to the invention can contain water in a quantity of up to 99.999999% by weight, preferably from 5 to 80% by weight, based on the total weight of the preparation.

The preparations according to the invention, comprising one or more of the compounds of formula (I) to be used according to the invention and/or salts of compounds of formula (I) are preferably produced in that the compound(s) of formula (I) and/or the salt(s) of one or more such compounds is/are worked into a corresponding preparation, i.e. in particular a preparation used for nutrition, oral care or enjoyment or a pharmaceutical (base) preparation intended for oral use with a solid or liquid carrier in the form of a solution or a mixture. Preparations according to the invention present as a solution can advantageously also be converted into a solid preparation by spray-drying.

According to a further preferred embodiment, to produce preparations according to the invention, the compounds of formula (I) to be used according to the invention and/or salts of such compounds as well as optionally further ingredients of the preparation according to the invention are previously (i.e. before being worked into the preparation) worked into emulsions, liposomes, for example starting from phosphatide choline, microspheres, nanospheres or also into capsules, granules or extruded material from a matrix suitable for foodstuffs and indulgence foods, for example of starch, starch derivatives, cellulose or cellulose derivatives (for example hydroxypropyl cellulose), other polysaccharides (for example alginate), natural fats, natural waxes (for example beeswax, carnauba wax) or of proteins, for example gelatin.

In a further preferred production process, the compounds of formula (I) and/or salts thereof are previously complexed with one or more suitable complex formers, for example with cyclodextrins or cyclodextrin derivatives, preferably α- or β-cyclodextrin and then used in this complexed form.

A preparation according to the invention is particularly preferred in which the matrix is selected such that the compound(s) of formula (I) and/or the salt(s) of such a compound are released in a delayed manner from the matrix so that a long-lasting effect is obtained.

Conventional basic substances, auxiliaries and additives for foodstuffs or indulgence foods as further ingredients in preparations according to the invention used for nutrition or enjoyment can be contained in a preparation according to the invention (as described above) or can be used for the production of such preparations, for example water, mixtures of fresh or processed, animal or vegetable basic substances or raw materials (for example raw, roast, dried, fermented, smoked and/or boiled meat, bone cartilage, fish, vegetables, fruits, herbs, nuts, vegetable or fruit juices or pastes or mixtures thereof), digestible or indigestible carbohydrates (for example sucrose, maltose, fructose, glucose, dextrins, amylose, amylopectin, inulin, xylanes, cellulose, tagatose), sugar alcohols (for example sorbitol, erythritol), natural or hardened fats (for example tallow, lard, palm oil, coconut butter, hardened vegetable fat), oils (for example sunflower oil, peanut oil, corn oil, olive oil, fish oil, soya bean oil, sesame oil), fatty acids or the salts thereof (for example potassium stearate), proteinogenic or non-proteinogenic amino acids and related compounds (for example γ-amino butyric acid, taurin), peptides (for example glutahthion), native or processed proteins (for example gelatin), enzymes (for example peptidases), nucleic acids, nucleotides, further taste correctors or taste-modulating substances for unpleasant taste sensations or not unpleasant taste sensations, in particular taste-modulating substances (for example inositol phosphate, nucleotides such as guanosine monophosphate, adenosine monophosphate or other substances such as sodium glutamate or 2-phenoxypropionic acid), emulsifiers (for example lecithins, diacylglycerols, gum arabic), stabilizers (for example carrageenan, alginate), preservatives (for example benzoic acid, sorbic acid), antioxidants (for example tocopherol, ascorbic acid), gelators (for example citric acid), organic or inorganic acidulants (for example malic acid, acetic acid, citric acid, tartaric acid, phosphoric acid), (optionally further) bitter principles (for example quinine, caffeine, limonine, amarogentin, humolones, lupolones, catechins, tannins), (optionally further) sweeteners (for example saccharin, cyclamate, aspartame, neotame), mineral salts (for example sodium chloride, potassium chloride, magnesium chloride, sodium phosphates), substances preventing enzymatic browning (for example sulfite, ascorbic acid), ethereal oils, plant extracts, natural or synthetic dyes or colored pigments (for example carotenoids, flavonoids, anthocyans, chlorophyll and derivatives thereof), spices, trigeminally effective substances or plant extracts containing such trigeminally effective substances, synthetic, natural or nature identical flavoring or aromatic substances as well as odor correctors.

Dental care products (as a base for preparations according to the invention used for oral care) generally comprise an abrasive system (abrasive or polishing agent), for example silicic acids, calcium carbonates, calcium phosphates, aluminum oxides and/or hydroxylapatites, surface-active substances, for example sodium lauryl sulfate, sodium lauryl sarcosinate and/or cocamidopropylbetaine, humectants, for example glycerol and/or sorbitol, thickening agents, for example carboxymethyl cellulose, polyethylene glycols, carrageenan and/or Laponite®, (optionally additional) sweeteners, for example saccharin, taste correctors for unpleasant further taste correctors for unpleasant taste sensations or normally not unpleasant taste sensations, taste-modulating substances (for example inositol phosphate, nucleotides such as guanosine monophosphate, adenosine monophosphate or other substances such as sodium glutamate or 2-phenoxypropionic acid), cooling active ingredients, for example menthol, menthol derivatives, (for example L-menthol, L-menthyllactate, L-menthylalkylcarbonates, menthone ketals, menthane carboxylic acid amides), 2,2,2-trialkylacetic acid amides (for example 2,2-diisopropylpropionic acid methyl amide), icilin derivatives, stabilizers and active ingredients, for example sodium fluoride, sodium monofluorophosphate, tin difluoride, quaternary ammonium fluorides, zinc citrate, zinc sulfate, tin pyrophosphate, tin dichloride, mixtures of various pyrophosphates, triclosan, cetylpyridinium chloride, aluminum lactate, potassium citrate, potassium nitrate, potassium chloride, strontium chloride, hydrogen peroxide, flavorings and/or sodium bicarbonate or taste correctors.

Chewing gums (as a further example of preparations according to the invention used for oral care) generally comprise a chewing gum base, i.e. a chewable mass which becomes malleable while being chewed, different types of sugar, sugar substitutes, sweeteners, sugar alcohols, taste correctors or taste modulators for unpleasant or generally not unpleasant taste sensations, taste-modulating substances (for example inositol phosphate, nucleotides such as guanosine monophosphate, adenosine monophosphate or other substances such as sodium glutamate or 2-phenoxypropionic acid), humectants, thickeners, emulsifiers, flavorings and stabilizers or odor correctors.

All the active ingredients, basic substances, auxiliaries and additives usually used for pharmaceutical preparations intended for oral use can be used as ingredients for oral pharmaceutical preparations according to the invention. The active ingredients used can be in particular unpleasant-tasting orally formulatable pharmaceutical active ingredients, in particular bitter-tasting substances, the bitter taste sensation of which can be masked or reduced according to the invention. The active ingredients, basic substances, auxiliaries and additives can be converted in a manner known per se into the application forms suitable for oral use. This is routinely carried out using inert, nontoxic, pharmaceutically suitable auxiliaries. These include, inter alia, carriers (for example microcrystalline cellulose), solvents (for example liquid polyethylene glycols), emulsifiers (for example sodium dodecylsulfate), dispersing agents (for example polyvinylpyrrolidone), synthetic and natural biopolymers (for example albumin), stabilizers (for example antioxidants such as ascorbic acid), colorings (for example inorganic pigments such as iron oxides) and odor correctors as well as taste correctors, in particular those which do not affect the bitter taste.

Preparations according to the invention (as described above) can preferably also contain a flavoring composition (not according to the invention) in order to (further) complete and refine the taste and/or odor of the preparation. Suitable flavoring compositions contain, for example synthetic, natural or nature-identical flavorings, fragrances and taste-imparting substances as well as suitable auxiliaries and carriers. In this respect, it is considered to be particularly advantageous that a bitter or metallic taste sensation arising from flavorings and fragrances or taste-imparting substances contained in the preparations according to the invention can be masked or reduced, thereby improving the overall flavor or taste profile.

Preparations according to the invention which are present as semi-finished products can be used to mask or reduce an unpleasant taste sensation of finished product preparations which are produced using the semi-finished preparation.

In a particularly preferred embodiment of the present invention, the compounds of formula (I) to be used according to the invention or the salts thereof are used in a preparation according to the invention (as described above) combined with at least one further substance for altering, masking or reducing an unpleasant taste sensation and/or for enhancing a pleasant taste sensation, the pleasant taste sensation preferably being a sweet and/or umami taste. In this way, it is possible to achieve a particularly efficient masking effect.

Further substances for masking or reducing an unpleasant taste sensation and/or for enhancing a pleasant taste sensation or taste correctors—without restricting the present invention thereto—are preferably selected from the group consisting of nucleotides (for example adenosine-5′-monophosphate, cytidine-5′-monophosphate) or the pharmaceutically acceptable salts thereof, lactisoles, sodium salts (for example sodium chloride, sodium lactate, sodium citrate, sodium acetate, sodium gluconoate), hydroxyflavanones, for example eriodictyol, sterubin (eriodictyol-7-methylether), homoeriodictyol, and the sodium, potassium, calcium, magnesium or zinc salts thereof (in particular those as described in EP 1 258 200, which is part of this application by way of reference with respect to the corresponding compounds disclosed therein), hydroxybenzoic acid amides, for example 2,4-dihydroxybenzoic acid vanillylamide, 2,4-dihydroxybenzoic acid-N-(4-hydroxy-3-methoxybenzyl)amide, 2,4,6-trihydroxybenzoic acid-N-(4-hydroxy-3-methoxybenzyl)amide, 2-hydroxy-benzoic acid-N-4-(hydroxy-3-methoxybenzyl)amide, 4-hydroxybenzoic acid-N-(4-hydroxy-3-methoxybenzyl)amide, 2,4-dihydroxybenzoic acid-N-(4-hydroxy-3-methoxybenzyl)amide-mono-sodium salt, 2,4-dihydroxybenzoic acid-N-2-(4-hydroxy-3-methoxyphenyl)ethylamide, 2,4-dihydroxybenzoic acid-N-(4-hydroxy-3-ethoxybenzyl)amide, 2,4-dihydroxybenzoic acid-N-(3,4-dihydroxybenzyl)amide and 2-hydroxy-5-methoxy-N-[2-(4-hydroxy-3-methoxyphenyl)ethyl]amide; 4-hydroxybenzoic acid vanillylamide (in particular those as described in WO 2006/024587, which is part of this application by way of reference with respect to the corresponding compounds disclosed therein); hydroxydeoxybenzoins, for example 2-(4-hydroxy-3-methoxyphenyl)-1-(2,4,6-trihydroxyphenyl)ethanone, 1-(2,4-dihydroxyphenyl)-2-(4-hydroxy-3-methoxyphenyl)ethanone, 1-(2-hydroxy-4-methoxyphenyl)-2-(4-hydroxy-3-methoxyphenyl)ethanone) (in particular those as described in WO 2006/106023 which is part of this application by way of reference with respect to the corresponding compounds disclosed therein); hydroxyphenyl alkane dions, for example gingerdion-[2], gingerdion-[3], gingerdion-[4], dehydrogingerdion-[2], dehydrogingerdion-[3], dehydrogingerdion-[4]) (in particular those as described in WO 2007/003527 which is part of this application by way of reference with respect to the corresponding compounds disclosed therein); diacetyl trimers (in particular those as described in WO 2006/058893 which is part of this application by way of reference with respect to the corresponding compounds disclosed therein); gamma-aminobutyric acids (in particular those as described in WO 2005/096841 which is part of this application by way of reference with respect to the corresponding compounds disclosed therein); divanillins (in particular those as described in WO 2004/078302 which is part of this application by way of reference with respect to the corresponding compounds disclosed therein) and 4-hydroxydihydrochalcones (preferably as described in US 2008/0227867 A1, which is part of this application by way of reference with respect to the corresponding compounds disclosed therein), in this respect in particular phloretin and davidigenin, amino acids or mixtures of whey proteins with lecithins, hesperetin as disclosed in WO 2007/014879 which is part of this application by way of reference with respect to the corresponding compounds), 4-hydroxychalcones as disclosed in WO 2007/107598 which is part of this application by way of reference with respect to the corresponding compounds, or propylenephenyl glycosides (chavicolgylcosides) as described in EP 1 955 601 A1 which is part of this application by way of reference with respect to the corresponding compounds, pellitorin and derived flavoring compositions as described in U.S. Provisional Application 60/944,854 and in the patent applications based thereon, umami compounds as described in WO 2008/046895 and EP 1 989 944 A1 which are in each case part of this application by way of reference with respect to the corresponding compounds as well as umami compounds as described in US Provisional Application 60/984,023 or U.S. Provisional Application 61/061,273 and in the patent applications based thereon, which are part of this application by way of reference with respect to the corresponding compounds disclosed therein.

Combinations with homoeriodictyol and the sodium, potassium, calcium, magnesium or zinc salts thereof, divanillins, phloretin and/or hesperitin are particularly preferred.

A further aspect of the present invention relates to a process for synergistically enhancing the sweet taste of a sweet-tasting substance, comprising the following steps:

i) preparation of a sweet-tasting substance (A) or of a both sweet and bitter-tasting substance (C),
ii) preparation
of a compound of formula (I),
or
of a salt of a compound of formula (I)
or
of a mixture of two or more different compounds of formula (I), two or more different salts of compounds of formula (I) or one or more different compounds of formula (I) and one or more different salts of compounds of formula (I),
as respectively described above,
   and
   iii) blending the components prepared in steps i) and ii) as well as optionally further components into a mixture in a ratio to one another such that the sweet taste of substance (A) or (C) is synergistically enhanced.

A process (as described above) is preferred

(a) for synergistically enhancing the sweet taste
and
(b) for masking or reducing the bitter taste sensation
of a both sweet and bitter-tasting substance (C), comprising the steps:
i) preparation of a both sweet and bitter-tasting substance (C),
ii) preparation
of a compound of formula (I),
or
of a salt of a compound of formula (I)
or
of a mixture of two or more different compounds of formula (I), two or more different salts of compounds of formula (I) or one or more different compounds of formula (I) and one or more different salts of compounds of formula (I),
as respectively described above,
   and
   iii) blending the components prepared in steps i) and ii) as well as optionally further components into a mixture in a ratio to one another such that (a) the sweet taste of the both sweet and bitter-tasting substance (C) is synergistically enhanced and (b) the bitter taste sensation of the both sweet and bitter-tasting substance (C) is masked or reduced.

The information provided above applies accordingly to further preferred embodiments of a process according to the invention, particularly in respect of the preferred selection of the substances (A), (B) and (C) and of the compounds of formula (I) or the salts thereof and mixtures thereof.

The invention will be further described in the following on the basis of examples. The examples serve to illustrate the invention, without thereby restricting the scope of protection of the claims. All numerical information relates to weight, unless stated otherwise.

### EXAMPLES

### General Procedure AAV 1

### Reduction of Flavonones into the Corresponding Flavans

The flavanone is introduced into pyridine (1.0 ml/mmol flavanone) and mixed with hexamethyldisilazane (1.0 ml/mmol flavanone). Trimethylchlorosilane (0.6 ml/mmol flavanone) is then added dropwise and the mixture is stirred for 30 minutes at room temperature. The volatile constituents are distilled off under reduced pressure and the residue is taken up in toluene (10.0 ml/mmol flavanone). After the insoluble constituents have been filtered off, the toluene is distilled off again under reduced pressure and the residue is taken up in THF (2.5 ml/mmol flavanone). The reaction solution is mixed with lithium borohydride (0.5 mmol/mmol flavanone). After stirring for a further hour at room temperature, 0.5 mg methylorange and sodium cyanoborohydride (1 mmol/mmol flavanone) is added. The mixture is then slowly titrated in 1N hydrochloric acid with the evolution of gas until there is a change in color (red) and the mixture is kept in this pH range by the further slow addition of hydrochloric acid. After approximately two hours and an addition of approximately 1.0 to 1.5 ml 1N hydrochloric acid per mmol flavanone, the red coloration remains permanently and the reaction is stirred for a further 12 hours at room temperature. The THF is distilled off again under reduced pressure and the residue is extracted with ethyl acetate. The organic phase is washed with 1N hydrochloric acid, distilled water and saturated sodium chloride solution, dried over sodium sulfate and concentrated. The product is purified by crystallization (ethyl acetate/hexane) and/or column chromatography (ethyl acetate/pentane).

### Example 1

### 3′,7-dihydroxy-4′-methoxyflavan (Compound 2)

### a) 1-[2-hydroxy-4-(tetrahydro-pyran-2-yloxy)-phenyl]-3-[4-methoxy-3-(tetrahydro-pyran-2-yloxy)-phenyl]-propenone

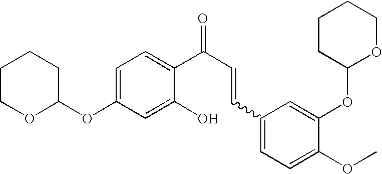

A solution of 25.65 g (109.6 mmol) 1-[2-hydroxy-4-(tetrahydro-pyran-2-yloxy)-phenyl]-ethanone and 25.56 g (108.2 mmol) 4-methoxy-3-(tetrahydro-pyran-2-yloxy)-benzaldehyde in 250 ml methanol is mixed with 8.25 g (122.9 mmol) of an 85% caustic potash solution and heated under reflux for 16 hours. After cooling to room temperature, the solution is mixed with 9.0 g (149.9.mmol) glacial acetic acid and the reaction mixture is concentrated on a rotary evaporator.

The residue is taken up in 300 ml ethyl acetate and 200 ml of water, the organic phase is separated and washed again with 200 ml of water, dried over sodium sulfate and concentrated to dryness. The aldol condensation product was used in the next step without being further purified (see b)).

### b) 7-hydroxy-2-(3-hydroxy-4-methoxy-phenyl)-chroman-4-one

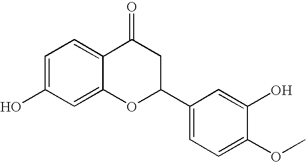

54.0 g of the above-mentioned (see a)) aldol condensation product are taken up in 300 ml of methanol and mixed with 10 ml of 37% hydrochloric acid. After the reaction mixture has been stirred for a further 14 hours at room temperature, the reaction batch is diluted with water to a volume of 1.5 liters, the corresponding deprotected chalcone separating out as a brown solid. This solid is taken up in ethyl acetate, dried over sodium sulfate and reconcentrated up to dryness.

The residue is dissolved in 250 ml of methanol and slowly mixed with 50 ml trifluoromethane sulfonic acid with ice cooling. After being stirred for a further three hours at room temperature, the reaction mixture is concentrated on a rotary evaporator and then taken up in 600 ml of ethyl acetate. The organic phase is washed twice with saturated sodium chloride solution, dried over sodium sulfate and concentrated on the rotary evaporator. The crude product weighed 19.1 g and, according to LC-MS, contained a mixture of the expected cyclic chromanone and the desired open-chain compound which is further reacted without purification (see c)).

### c) 3′,7-dihydroxy-4′-methoxyflavan (Compound 2)

19.1 g of the above-mentioned chromanone/chalcone mixture are dissolved in 200 ml THF and mixed with 12.57 (200.0 mmol) sodium cyanoborohydride. The reaction mixture is then adjusted to a pH range of from 2.8 to 3.2 by slowly adding 1N hydrochloric acid with the evolution of gas, this pH range being maintained by adding more 1N hydrochloric acid. When the pH of the reaction no longer changes, the mixture is subsequently stirred for a further 40 hours at room temperature. The THF is distilled off again under reduced pressure and the residue is extracted with ethyl acetate. The organic phase is washed with 1N hydrochloric acid, distilled water and saturated sodium chloride solution, dried over sodium sulfate and concentrated. The crude product is taken up again in ethyl acetate, filtered over 90 g silica and then concentrated. By recrystallization (ethyl acetate/hexane 1:2), 2.20 g (8.1 mmol) of the desired flavan derivative (compound 2) is finally obtained. Compound 2 is present as a racemic mixture. The same preferably applies to the compounds of formula (I) which are prepared in Examples 3 to 8 and are to be used according to the invention.

¹H-NMR (400 MHz, CDCl₃): 2.03 (m, 1H); 2.15 (m, 1H); 2.71 (m, 1H); 2.88 (m, 1H); 3.89 (s, 3H); 4.67 (s, 1H); 4.95 (dd, J=2.6 Hz, J=10.1 Hz, 1H); 5.63 (s, 1H); 6.38 (dd, J=2.6 Hz, J=9.0 Hz, 1H); 6.38 (d, J=2.3 Hz, 1H); 6.85 (d, J=8.3, 1H); 6.89-6.94 (kB, 2H); 6.99 (d, J=2.1 Hz, 1H) ppm.

¹³C-NMR (100 MHz, DMSO): 23.5 (CH₂); 29.3 (CH₂); 55.6 (CH₃); 76.5 (CH); 102.6 (CH); 107.8 (CH); 111.9 (CH); 112.1 (C); 113.3 (CH); 116.7 (CH); 129.7 (CH); 132.2 (C); 146.3 (C); 147.0 (C); 155.3 (C); 156.4 (C) ppm.

Mass spectrum (EI): m/z (%)=273 (17); 272 (M^{·+}, 100); 163 (14); 162 (31); 150 (87); 137 (38); 135 (48); 123 (14); 107 (17); 77 (14).

### Example 2

### (2S)-3′,7-dihydroxy-4″-methoxyflavan (Compound S-2)

The compound was isolated analogously to Masaoud et al., (1995) “Flavonoids of dragon's blood from Dracaena cinnabari”, Phytochemistry, Volume 38, 745-749 from “Dragons blood”. The amount of rotation of the sample was [ ]_{D}²⁴=45° (MeOH, c=0.30). According to chiral GC, the sample was present to >90% as (2S)-enantiomer (column DAcTBS- -CD, carrier gas 2 mL H₂/min, temperature program: 100-4-220° C., RT 54 min, comparison with the racemic sample from Example 1). The MS and NMR data is identical to the sample from Example 1.

### Example 3

### 4′,7-dihydroxy-3″-methoxyflavan (Compound 3)

### a) (E)-1-(4-benzyloxy-3-methoxy-phenyl)-3-(2,4-bis-benzyloxy-phenyl)-propenone

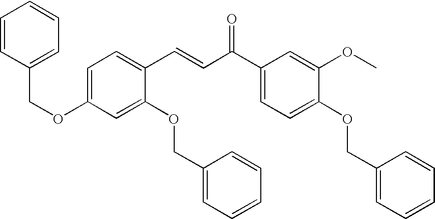

A solution of 6.7 g (21.0 mmol) 2,4-bis-benzyloxybenzaldehyde and 5.4 g (21.1 mmol) 1-(4-benzyloxy-3-methoxy-phenyl)ethanone in 125 ml ethanol are mixed with 0.69 g (10.5 mmol) caustic potash and heated for 15 hours under reflux, the product already separating out. After the end of the reaction, 50 ml of water and 300 ml of ethanol are added and the mixture is reheated under reflux until all of the solid material is dissolved again. After cooling, the solid material which has now separated out again is filtered off, washed with sufficient water and then dried at 70° C. The crude product weighs 10.5 g and is used in the next step without being further purified (see b)).

### b) 4-[3-hydroxy-3-(4-hydroxy-3-methoxy-phenyl)-Propyl]-benzene-1,3-diol

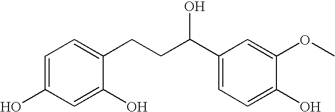

10.5 g of the above-mentioned crude product (see a)), i.e. of the aldol condensation product are taken up in 150 ml of dry THF and cooled to 10° C. under a nitrogen atmosphere. 0.36 g (9.5 mmol) lithium aluminum hydride are then quickly added and, in so doing, the internal temperature, i.e. the temperature in the reaction flask, should not exceed 20° C. The mixture is then stirred for five hours at room temperature before the reaction mixture is cooled to 0° C. and mixed in succession with 0.4 ml of water, 0.4 ml of 15% sodium hydroxide solution and 1.2 ml of water. 20.0 g of sodium sulfate are then added and the mixture is stirred for a further 30 minutes. Finally, the solids are filtered off and the solution is concentrated on the rotary evaporator.

The intermediate product is taken up in a solvent mixture of 100 ml ethanol and 100 ml ethyl acetate and after the addition of 3.0 g hydration catalyst (5% palladium on carbon), is stirred for 9 hours at room temperature under a hydrogen atmosphere, 950 ml of hydrogen being consumed. The catalyst is separated by filtration over kieselguhr and, after removing the solvent, 3.94 g of the desired, deprotected, open-chain compound is obtained, which is used without being purified in the subsequent cyclization (see c)).

### c) 4′,7-dihydroxy-3″-methoxyflavan (Compound 3)

3.94 g of the deprotected, open-chain compound described above (see b)) are dissolved in 100 ml THF and heated with 5.0 ml of 85% phosphoric acid for four hours under reflux. The reaction mixture is then cooled to room temperature and mixed with 350 ml ethyl acetate and 200 ml water with stirring. The organic phase is separated and washed with 100 ml of saturated sodium chloride solution. After removing the solvent and subsequent column chromatography on silica (ethyl acetate/hexane 1:1), 1.36 g (5.0 mmol) of the desired 4′,7-dihydroxy-3″-methoxyflavan (compound 3) are obtained as a colorless to faint pink solid material.

¹H-NMR (400 MHz, CDCl₃): 2.05 (m, 1H); 2.15 (m, 1H); 2.73 (m, 1H); 2.91 (m, 1H); 3.90 (s, 3H); 4.71 (s, 1H); 4.95 (dd, J=2.6 Hz, J=10.3 Hz, 1H); 5.62 (m, 1H); 6.37-6.41 (kB, 2H); 6.88-6.95 (kB, 4H) ppm.

¹³C-NMR (100 MHz, CDCl₃): 24.6 (CH₂); 30.1 (CH₂); 55.9 (CH₃); 78.0 (CH); 103.5 (CH); 107.9 (CH); 108.7 (CH); 114.2 (C); 114.3 (CH); 119.2 (CH); 130.2 (CH); 133.6 (C); 145.4 (C); 146.6 (C); 154.8 (C); 156.0 (C) ppm.

Mass spectrum (EI): m/z (%)=273 (14); 272 (M^{·+}, 61); 151 (12); 150 (100); 137 (34); 135 (32); 123 (17); 122 (16); 107 (13); 28 (38).

### Example 4

### 3′,4′,5-trihydroxy-7-methoxyflavan (Compound 13)

According to AAV 1, 4.00 g (13.3 mmol) of sterubin were converted into 0.28 g (1.0 mmol) of the corresponding flavan or flavan derivative (compound 13).

¹H-NMR (400 MHz, DMSO): 1.83 (m, 1H); 2.02 (m, 1H); 2.54 (m, 2H); 3.62 (s, 3H); 4.79 (dd, J=2.1 Hz, J=10.0 Hz, 1H); 5.86 (d, J=2.5 Hz, 1H); 5.98 (d, J=2.5 Hz, 1H); 6.64 (dd, J=2.1 Hz, J=6.7 Hz, 1H); 6.71 (d, J=8.1, 1H); 6.78 (d, J=2.0, 1H); 8.6-9.4 (kB, 3H) ppm.

¹³C-NMR (100 MHz, DMSO): 18.9 (CH₂); 28.8 (CH₂); 54.7 (CH₃); 76.6 (CH); 92.4 (CH); 93.7 (CH); 101.8 (C); 113.6 (CH); 115.1 (CH); 117.1 (CH); 132.4 (C); 144.7 (C); 144.9 (C); 156.1 (C); 156.3 (C); 158.4 (C) ppm.

Mass spectrum (EI): m/z (%)=288 (M^{·+}, 28); 192 (9); 165 (21); 153 (100); 152 (7); 148 (19); 140 (10); 137 (9); 136 (11); 65 (8).

### Example 5

### 4′,5,7-trihydroxyflavan (Compound 14)

According to AAV 1, 2.36 g (8.7 mmol) naringenin were converted into 1.02 g (3.9 mmol) of the corresponding flavan or flavan derivative (compound 14).

¹H-NMR (400 MHz, CD₃OD): 1.91 (m, H); 2.08 (m, 1H); 2.58 (m, 1H); 2.68 (m, 1H); 4.81 (dd, J=2.0 Hz, J=10.3 Hz, 1H); 5.85 (d, J=2.3 Hz, 1H); 5.93 (d, J=2.3 Hz, 1H); 6.78 (m, 2H); 7.21 (m, 2H); 8.6-9.4 (kB, 3H) ppm.

¹³C-NMR (100 MHz, CD₃OD): 20.5 (CH₂); 30.8 (CH₂); 78.9 (CH); 95.98 (CH); 96.05 (CH); 102.6 (C); 116.1 (CH); 128.5 (CH); 134.3 (C); 157.3 (C); 157.4 (C); 158.00 (C); 158.03 (C) ppm.

Mass spectrum (EI): m/z (%)=269 (18); 258 (M^{·+}, 77); 152 (16); 139 (94); 133 (29); 120 (100); 107 (28); 91 (29); 55 (16); 28 (33).

### Example 6

### 3′,5,7-trihydroxy-4′-methoxyflavan (Compound 15)

According to AAV 1, 3.02 g (10.0 mmol) hesperitin were converted into 1.74 g (6.0 mmol) of the corresponding flavan or flavan derivative (compound 15).

¹H-NMR (400 MHz, DMSO): 1.81 (m, 1H); 2.02 (m, 1H); 2.46-2.54 (kB, 2H); 3.76 (s, 3H); 4.81 (dd, J=2.1 Hz, J=9.4 Hz, 1H); 5.70 (d, J=2.3 Hz, 1H); 5.89 (d, J=2.3 Hz, 1H); 6.76 (ddd, J=0.6 Hz, J=2.1 Hz, J=6.8 Hz, 1H); 6.81 (d, J=2.1, 1H); 6.89 (d, J=8.4, 1H); 8.91 (s, 1H); 8.97 (s, 1H); 9.16 (s, 1H) ppm.

¹³C-NMR (100 MHz, DMSO): 18.7 (CH₂); 28.9 (CH₂); 55.6 (CH₃); 76.2 (CH); 94.2 (CH); 94.9 (CH); 100.1 (C); 111.9 (CH); 113.3 (CH); 116.8 (CH); 134.4 (C); 146.2 (C); 147.0 (C); 155.9 (C); 156.0 (C); 156.2 (C) ppm.

Mass spectrum (LC-MS: C18; 100×2.1 mm; 0.2 ml/min; H₂O/CH₃CN 100/0→0/100): m/z (%)=334 (15); 333 (100); 331 (8); 289 (5); 288 (34).

### Example 7

### 4′,5,7-trihydroxy-3′-methoxyflavan (Compound 16)

According to AAV 1, 3.02 g (10.0 mmol) homoeriodyctiol were converted into 0.70 g (2.4 mmol) of the corresponding flavan or flavan derivative (compound 16).

¹H-NMR (400 MHz, DMSO): 1.87 (m, 1H); 2.03 (m, 1H); 2.43-2.59 (kB, 2H); 3.77 (s, 3H); 4.80 (dd, J=2.0 Hz, J=10.2 Hz, 1H); 5.69 (d, J=2.3 Hz, 1H); 5.88 (d, J=2.3 Hz, 1H); 6.75 (d, J=6.8 Hz, 1H); 6.79 (dd, J=1.9 Hz, J=6.8 Hz, 1H); 6.94 (d, J=1.9, 1H); 8.90 (bs, 1H); 8.93 (bs, 1H); 9.15 (s, 1H) ppm.

¹³C-NMR (100 MHz, DMSO): 19.1 (CH₂); 28.9 (CH₂); 55.5 (CH₃); 76.6 (CH); 94.2 (CH); 94.9 (CH); 100.1 (C); 110.4 (CH); 115.0 (CH); 118.6 (CH); 132.6 (C); 145.9 (C); 147.3 (C); 156.0 (C); 156.16 (C); 156.18 (C) ppm.

Mass spectrum (LC-MS: C18; 100×2.1 mm; 0.2 ml/min; H₂O/CH₃CN 100/0→0/100): m/z (%)=342 (3); 334 (7); 330 (3); 289 (6); 288 (100).

### Example 8

### 4′,7-dihydroxyflavan (Compound 1)

### a) (E)-1-(4-benzyloxy-phenyl)-3-(2,4-bis-benzyloxyphenyl)-propenones

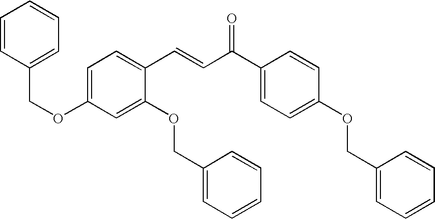

A solution of 31.8 g (0.1 mol) 2,4-bis-benzyloxybenzaldehyde and 22.6 g (0.1 mol) 1-(4-benzyloxy-phenyl)ethanone in 300 ml ethanol was mixed with 6.6 g (0.1 mol; 85% in H₂O) caustic potash and heated for 8 hours under reflux, the product already separating out. After the end of the reaction, the solid material is filtered off, the filter residue is washed four times with in each case 75 ml of a 1:1 mixture of water and ethanol and then dried at 70° C. The crude product weighs 48.8 g and is used in the next step without further purification (see b)).

### b) 4-[3-hydroxy-3-(4-hydroxy-phenyl)-Propyl]-benzene-1,3-diol

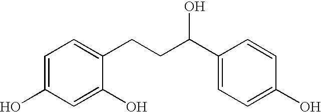

21.0 g of the above-mentioned crude product (see a)), i.e. of the aldol condensation product are taken up in 100 ml of dry THF and 300 ml of dry diethyl ether and cooled to 10° C. under a nitrogen atmosphere. 0.76 g (20.0 mmol) lithium aluminum hydride are then quickly added and, in so doing, the internal temperature, i.e. the temperature in the reaction flask, should not exceed 20° C. The mixture is then stirred for five hours at room temperature before the reaction mixture is again cooled to 10° C. and mixed with 10% hydrochloric acid and ice water. After the organic phase has been separated, it is washed with water and saturated sodium hydrogen carbonate solution, dried over sodium sulfate and concentrated on the rotary evaporator. The intermediate product is taken up in a solvent mixture of 300 ml isopropanol and 100 ml tetrahydrofuran and after the addition of 5.0 g hydration catalyst (5% palladium on carbon), is stirred for 8 hours at 35° C. under a hydrogen atmosphere, 2950 ml of hydrogen being consumed. The catalyst is separated by filtration over kieselguhr and, after removing the solvent, 20.4 g of the desired, deprotected, open-chain compound is obtained, which is used without being purified in the subsequent cyclization (see c)).

### c) 4′,7-dihydroxyflavan (Compound 1)

20.4 g of the deprotected, open-chain compound (see b)) described above are dissolved in 400 ml THF and heated with 10.0 ml of 85% phosphoric acid for six hours under reflux. The reaction mixture is then cooled to room temperature and mixed with 500 ml ethyl acetate and 1000 ml water. The organic phase is separated and washed twice with in each case 300 ml of water and dried over sodium sulfate. After removing the solvent and subsequent column chromatography on silica (ethyl acetate/hexane 1:1), 2.57 g (10.6 mmol) of the desired 4′,7-dihydroxyflavan (compound 1) are obtained as a colorless to faint pink solid material.

¹H-NMR (400 MHz, DMSO): 1.92 (m, 1H); 2.03 (m, 1H); 2.59 (m, 1H); 2.80 (m, 1H); 4.90 (dd, J=2.2 Hz, J=10.0 Hz, 1H); 6.17 (d, J=2.4 Hz, 1H); 6.27 (dd, J=2.5 Hz, J=8.2 Hz, 1H); 6.75 (m, 2H); 6.86 (d, J=2.5 Hz, 1H); 7.20 (m, 2H); 9.14 (bs, 1H); 9.38 (bs, 1H) ppm.

¹³C-NMR (100 MHz, DMSO): 23.8 (CH₂); 29.2 (CH₂); 767 (CH); 102.6 (CH); 107.8 (CH); 1121 (C); 114.9 (CH); 127.3 (CH); 129.7 (CH); 131.8 (C); 155.4 (C); 156.3 (C); 156.8 (C) ppm.

Mass spectrum (LC-MS: EclipseC18; 0.2 ml/min; H₂O/CH₃CN 100/0→5/95, water contains 0.01% formic acid): m/z (%)=242 (12); 241 (68); 239 (5); 163 (5); 135 (39); 122 (8); 121 (100); 119 (77).

### Application Example 1

### Bitter Reduction of a Bitter Substance Solution

To quantify the reduction (i.e. the masking or reduction) of the bitter sensation in a sample, the bitterness of a solution containing 500 ppm of caffeine was compared by a panel of experts in each case with a sample which contained 500 ppm of caffeine and additionally 50 ppm or 100 ppm of a substance to be assessed (in respect of the bitter-reduction ability) (grading: 1 [not bitter] to 10 [extremely bitter]).

For the evaluation, i.e. the calculation of the reduction (in %) of the bitter sensation, the mean values of the assessments by the panel of experts of the caffeine solution and the sample, to be compared, containing caffeine and a substance to be assessed were used in each case. In this respect, 2,4-dihydroxybenzoic acid (2,4-DHB) was used as the comparison (reference) according to U.S. Pat. No. 5,643,941.

| | | Bitter sensation | | | Observations |
|---|---|---|---|---|---|
| | | (1-10) | | Reduction | on the |
| | Caffeine | | Sample | of the | sensory |
| (Test/comparative) | (bitter | Caffeine | (caffeine + | bitter | analysis of the |
| substance | substance) | solution | substance) | sensation | sample |
| 100 ppm 2,4-DHB | 500 ppm | 5.1 ± 1.0 | 5.0 ± 1.0 | 3% | — |
| 50 ppm 7,4′-dihydroxy-3′- | 500 ppm | 5.4 ± 1.5 | 3.9 ± 1.9 | 27% | Sweet, |
| methoxyflavan | | | | (p < 0.05) | alcoholic, |
| (compound 2 from Example | | | | | liquorice |
| 1) | | | | | |
| 50 ppm (2S)-7,4′-dihydroxy- | 500 ppm | 4.7 ± 1.6 | 3.9 ± 1.5 | 16% | — |
| 3′-methoxyflavan | | | | | |
| (compound S-2 from | | | | | |
| Example 2) | | | | | |
| 50 ppm 3′,7-dihydroxy-4′- | 500 ppm | 4.8 ± 2.3 | 4.1 ± 1.4 | 15% | Slightly tart, |
| methoxyflavan | | | | | sweet |
| (compound 3 from Example | | | | | |
| 3) | | | | | |
| 50 ppm 4′,5,7- | 500 ppm | 5.1 ± 2.1 | 3.8 ± 1.9 | 26% | ethanolic, fruity, |
| trihydroxyflavan | | | | (p < 0.05) | green |
| (compound 14 from | | | | | |
| Example 5) | | | | | |
| 50 ppm 3′,5,7-trihydroxy-4′- | 500 ppm | 4.4 ± 1.6 | 3.8 ± 1.5 | 15% | — |
| methoxyflavan | | | | | |
| (compound 15 from | | | | | |
| Example 6) | | | | | |
| 50 ppm 4′,5,7-trihydroxy-3′- | 500 ppm | 4.4 ± 1.6 | 4.1 ± 2.0 | 5% | smoother, |
| methoxyflavan | | | | | slightly |
| (compound 16 from | | | | | alcoholic |
| Example 7) | | | | | |

### Application Example 2

### Enhancement of the Sweet Sensation of a Sugar Solution

### Comparative Test 1:

To quantify the enhancement of the sweet sensation, the sweetness of a 5% sucrose solution was compared by a panel of experts in each case with a sample which contained 5% of sucrose as well as 50 ppm or 100 ppm of a compound of formula (I) to be used according to the invention (grading: 1 [not sweet] to 10 [extremely sweet]).

For the evaluation, i.e. the calculation of the enhancement (in %) of the sweet sensation, the mean values of the assessments by the panel of experts of the sucrose solution and the sample, to be compared, containing sucrose and a compound of formula (I) were used in each case.

| | | Sweet sensation (1-10) | | |
|---|---|---|---|---|
| (Test/ | | | Sample | Enhancement |
| comparative) | Sucrose | Sucrose | (sucrose + | of the sweet |
| substance | (sweetener) | solution | substance) | sensation |
| 50 ppm | 5% | 5.1 ± 0.9 | 8.1 ± 1.3 | 57% |
| compound 2 | | | | (p < 0.001) |
| Example1) | | | | |
| 50 ppm | 5% | 5.7 ± 1.2 | 7.8 ± 1.7 | 36% |
| compound S-2 | | | | (p < 0.001) |
| (Example 2) | | | | |
| 50 ppm | 5% | 4.8 ± 1.1 | 5.8 ± 1.6 | 21% |
| compound 15 | | | | (p < 0.005) |
| (Example 6) | | | | |
| 100 ppm | 5% | 5.1 ± 1.4 | 6.2 ± 1.5 | 21% |
| compound 1 | | | | (p < 0.05) |
| (Example 8) | | | | |

### Comparative Test 2:

The following comparative test was carried out to assess the synergistically sweet-enhancing effect of a compound of formula (I) to be used according to the invention:

The inherent sweetness of compound 2 from Example 1, which was dissolved in water in a pure form in different concentrations (0.0025 or 0.0050% by weight in water) was determined by a panel of experts (panelists) using a series of comparisons of different sucrose concentrations in water (0; 0.25; 0.5; 0.75; 1; 1.5; 2; 3; 4 and 5% by weight sucrose in water).

The panelists were asked to test each solution of compound 2 in water against the sucrose series and to grade them according to their degree of sweetness (sucrose equivalent). It was possible to determine the inherent sweetness of compound 2 for the above-mentioned concentrations from the results provided by the panelists. In addition to the sucrose equivalence in water (i.e. without sucrose), the sucrose equivalence in a 5% sucrose solution was also determined.

| Concentration of | | Sucrose equivalence in a | |
|---|---|---|---|
| compound 2 from | Determination of the | 5% sucrose solution | |
| Example1 in water | sucrose equivalence in | Calculation | Determi- |
| [% by weight] | water (without sucrose) | (5 + x) % | nation |
| 0.0025 | 0.84% | 5.84% | 6.8% |
| 0.0050 | 1.45% | 6.45% | 8.2% |

It was possible to establish a clear synergistic sweetness enhancement even at a low concentration, not sweet per se, of approximately 25 ppm of compound 2 (from Example 1). From a purely calculational point of view, it was to be expected that a 5% sucrose solution (i.e. a sucrose solution which corresponds in sensory manner by definition to 5% sucrose equivalents) which contains 25 ppm or 50 ppm of compound 2 (from Example 1) produced a sweetness of approximately 5.84 or 6.45 sucrose equivalents by a purely additive effect of the inherent sweetness of compound 2 (see the determined sucrose equivalence in water). However, the sensory assessment of such a 5% sucrose solution with 25 ppm or 50 ppm of compound 2 (from Example 1) by the panelists produced an averaged sucrose equivalence of 6.8% or 8.2%. Considering the values to be expected from a purely calculational point of view, this corresponds to a synergy or a synergistic additional effect of approximately 17% or 27%.

### Application Example 3

### Spray-Dried Preparation as Semi-Finished Product for Flavoring Finished Products

| Ingredient | Amount used in % by weight | | | | | | | | |
|---|---|---|---|---|---|---|---|---|---|
| Preparation | A | B | C | D | E | F | G | H | I |
| Drinking water | 60.8 | 60.8 | 56.4 | 60.8 | 60.8 | 60.8 | 60.8 | 60.8 | 60.8 |
| Maltodextrin from wheat | 24.3 | 24.3 | 24.3 | 24.3 | 24.3 | 24.3 | 24.3 | 24.3 | 24.3 |
| Gum arabic | 6.1 | 6.1 | 6.1 | 6.1 | 6.1 | 6.1 | 6.1 | 6.1 | 6.1 |
| Compound 2 (Example 1) | 8.8 | — | — | — | 6.6 | 5.5 | 3.3 | 4.4 | — |
| Compound S-2 (Example 2) | — | 8.8 | — | — | — | — | — | — | — |
| Compound 14 (Example 5) | — | — | 13.2 | — | — | — | — | — | — |
| Compound 15 (Example 6) | — | — | — | 8.8 | — | — | — | — | — |
| Compound 1 (Example 8) | — | — | — | — | — | — | — | — | 6.6 |
| Hesperetin | — | — | — | — | 2.2 | — | — | 1.1 | — |
| Homoeriodictyol-sodium salt | — | — | — | — | — | — | 5.5 | 3.3 | — |
| Phloretin | — | — | — | — | — | 3.3 | — | — | 2.2 |

The drinking water is introduced into a container and maltodextrin and gum arabic are dissolved therein. The flavorings are then emulsified into the carrier solution using a Turrax. The temperature of the spray solution should not exceed 30° C. The mixture is then spray-dried (set temperature at inlet: 185 to 195° C., set temperature at outlet: 70 to 75° C.

### Application Example 4

### Combination with Sweeteners

90 g of sucrose and 10 g of tagatose are added to and mixed with 0.5 g of a spray-dried semi-finished product from Application Example 3 (according to preparation B). The product can be used, for example as a sweetener with a bitter masking effect for coffee or tea.

### Application Example 5

### Reduced-Sugar Soft Drink

Preparation A: comparative preparation with 10% sugar
Preparation B: comparative preparation with 8% sugar
Preparation C: comparative preparation with 7% sugar
Preparation D-G: preparations according to the invention with 7 or 8% sugar, reduced in sugar compared to A (and for preparations E-G compared to B)

| Ingredient | Amount used in % by weight | | | | | | |
|---|---|---|---|---|---|---|---|
| Preparation | A | B | C | D | E | F | G |
| Sugar | 10 | 8 | 7 | 8 | 7 | 7 | 7 |
| Citric acid | 0.15 | 0.15 | 0.15 | 0.15 | 0.15 | 0.15 | 0.15 |
| Lemon flavoring | 0.1 | 0.1 | 0.1 | 0.1 | 0.1 | 0.1 | 0.1 |
| Compound 2, | — | — | — | 0.005 | 0.005 | 0.005 | 0.005 |
| Example 1 | | | | | | | |
| Phloretin | — | — | — | — | — | 0.002 | — |
| Hesperetin | — | — | — | — | — | — | 0.001 |

| | |
|---|---|
| Water | ad 100 |

The ingredients were mixed in the stated sequence and made up to 100% by weight with water. The mixtures were poured into glass bottles and carbonated.

The preparations were then tested in a sensory manner in blind duo-tests and compared with one another. In this respect, the sweetness was assessed by experts using a grading of 1 [not sweet] to 10 [extremely sweet].

| Comparison | | | Difference | |
|---|---|---|---|---|
| (1st and 2nd | Sweetness sensation (1-10) | | (sweetness | Significance |
| samples) | 1. Probe | 2. Probe | sensation) | p |
| Preparations | 7.1 ± 1.4 | 4.6 ± 1.3 | −36% | <0.001 |
| A and B | | | | |
| Preparations | 6.3 ± 1.6 | 6.2 ± 1.9 | −1% | >0.95 |
| A and D | | | | |
| Preparations | 7.1 ± 1.8 | 4.5 ± 1.4 | −37% | <0.001 |
| A and C | | | | |
| Preparations | 7.1 ± 1.2 | 5.9 ± 1.6 | −16% | <0.05 |
| A and E | | | | |
| Preparations | 6.5 ± 1.6 | 6.0 ± 2.1 | −8% | =0.44 |
| A and F | | | | |
| Preparations | 6.5 ± 1.3 | 6.4 ± 2.2 | −1% | >0.95 |
| A and G | | | | |

Accordingly, a decrease in sweetness of approximately 36 or 37% (see table) was observed by reducing the amount of sugar (by 2 and respectively 3% by weight for preparation B and respectively preparation C, based on the total weight of the preparation).

By adding a small amount of a compound of formula (I) to be used according to the invention on its own (see preparations D and E) or combined with known flavorings for enhancing sweetness (see preparations F and G), in an ideal case (A versus D and A versus G) no relevant difference could be established between the full sugar preparation and the preparation according to the invention having less sugar, due to the synergistically sweet-enhancing effect of compound 2.

### Application Example 6

### Tea Preparation

| Ingredient | Amount used in % by weight | | |
|---|---|---|---|
| Preparation | A | B | C |
| black tea, Ceylon, leaf product | 94.00% | | |
| green tea, China, leaf product | | 92.00% | |
| Mate tea, Peru, leaf product | | | 95.00% |
| Semi-finished product A from | 6% | | |
| Application Example 3 | | | |
| Semi-finished product G from | | 8% | |
| Application Example 3 | | | |
| Semi-finished product H from | | | 5% |
| Application Example 3 | | | |

The tea and the semi-finished product are mixed and packaged into tea bags made of filter paper. For use, a tea bag is infused in 100-250 ml of boiling water and left to draw for 2-5 minutes.

### Application Example 7

### Use in a Soya Drink

Compound 2, Example 1, respectively compound S-2, Example 2, was pre-dissolved in ethanol and added to a soya milk from a local supermarket. The mixture was stirred together with a milk flavoring in a tumbler.

| Ingredient | Amount used in % by weight | | | |
|---|---|---|---|---|
| Preparation | A | B | C | D |
| Soya milk (local supermarket, | 96.7 | 99.68 | 98.29 | 97.60 |
| unflavored, unsweetened) | | | | |
| Vanilla flavoring | 0.1 | 0.1 | | 0.05 |
| Milk flavoring | | | 0.1 | 0.05 |
| Saccharose | 3 | | 1.5 | 2 |
| Rebaudioside A 95% | | 0.02 | 0.01 | |
| Emulgum | 0.1 | 0.1 | | 0.1 |
| 10% compound 2, Example 1 | 0.1 | | 0.1 | |
| in ethanol | | | | |
| 10% compound S-2, Example 2 | | 0.1 | | 0.1 |
| in ethanol | | | | |
| Hesperetin, 5% in ethanol | | | | 0.1 |

### Application Example 8

### Use in a Chewing Gum

| | | Amount used in % by |
|---|---|---|
| Part | Ingredient | weight |
| A | Chewing gum base, | 30.00 |
| | Company “Jagum T” | |
| B | Sorbitol, pulverized | 39.00 |
| | Isomalt ® | 9.50 |
| | (Palatinit GmbH) | |
| | Xylitol | 2.00 |
| | Mannitol | 3.00 |
| | Rebaudioside A 98% | 0.2 |
| | Emulgum ® | 0.30 |
| | (Colloides Naturels, Inc.) | |
| C | Sorbitol, 70% | 14.00 |
| | Glycerin | 1.00 |
| D | Flavoring, containing 1% by weight | 1.00 |
| | of compound 2, Example 1, based on | |
| | the total weight of the flavoring | |

Parts A to D are mixed and kneaded intensively. The raw mixture can be processed into ready-for-use chewing gum, for example as thin strips.

### Application Example 9

### Use in a Toothpaste

| | | Amount used in % by |
|---|---|---|
| Part | Ingredient | weight |
| A | Demineralized water | 22.00 |
| | Sorbitol (70%) | 45.00 |
| | Solbrol ® M, sodium salt (Bayer AG, p- | 0.15 |
| | Trisodium phosphate | 0.10 |
| | Rebaudioside A, 98% | 0.10 |
| | Sodium monofluorophosphate | 1.12 |
| | Polyethylene glycol 1500 | 5.00 |
| B | Sident 9 (abrasive silicon dioxide) | 10.00 |
| | Sident 22 S (thickening silicon dioxide) | 8.00 |
| | Sodium carboxymethylcellulose | 0.90 |
| | Titanium dioxide | 0.50 |
| C | Demineralized water | 4.53 |
| | Sodiumlauryl sulfate | 1.50 |
| D | Flavoring, containing 1% by weight | 1.00 |
| | of compound 2, Example 1, based on | |
| | the total weight of the flavoring | |

The ingredients of parts A and B are respectively pre-mixed per se and stirred together thoroughly for 30 minutes under vacuum at 25-30° C. Part C is pre-mixed and added to A and B; D is added and the mixture is stirred thoroughly for 30 minutes under vacuum at 25-30° C. After expanding, the toothpaste is ready and can be filled into a dispenser.

### Application Example 10

### Sugar-Free Hard Caramel

| | Content (%) | | | |
|---|---|---|---|---|
| Ingredient | A | B | C | D |
| Palatinite, type M | 75.00 | 74.00 | 75.50 | 75.00 |
| Citric acid | — | 1.0 | 0.5 | — |
| Water | 24.88 | 24.842 | 23.88 | 24.844 |
| Yellow coloring | — | 0.01 | — | — |
| Red coloring | — | — | 0.01 | — |
| Blue coloring | 0.01 | — | — | 0.01 |
| Peppermint flavoring | 0.1 | — | — | 0.1 |
| Lemon flavoring | — | 0.1 | — | — |
| Red fruit flavoring | — | — | 0.1 | — |
| Rebaudioside A 98% | — | 0.040 | — | 0.040 |
| 3′,7-dihydroxy-4′- | 0.010 | 0.005 | 0.010 | 0.005 |
| methoxyflavan (compound 2, | | | | |
| Example 1) | | | | |
| Hesperetin | — | 0.001 | — | 0.001 |
| Phloretin | — | 0.002 | — | — |

Palatinite was mixed with water and the mixture was melted at 165° C. and then cooled to 115° C. The remaining ingredients were added and after being thoroughly mixed, the mixture was poured into moulds, removed from the moulds after solidifying and then packaged individually.

### Application Example 11

### Reduced-Sugar Steamed Pudding

A: comparative preparation with 7.8% sucrose content
B: comparative preparation with reduced sucrose content (compared to A)
C: preparation according to the invention with reduced sucrose content (compared to A) and 3′,7-dihydroxy-4′-methoxyflavan (compound 2, Example 1)
D: preparation according to the invention with reduced sucrose content (compared to A), D-tagatose and 3′,7-dihydroxy-4′-methoxyflavan (compound 2, Example 1)

| | Preparation (amounts in % by weight) | | | |
|---|---|---|---|---|
| Ingredient | A | B | C | D |
| Sucrose | 7.8% | 5.4% | 5.4% | 5.4% |
| Starch | 3.0% | 3.0% | 3.0% | 3.0% |
| Skimmed milk | 1.5% | 1.5% | 1.5% | 1.5% |
| powder | | | | |
| Aubygel MR50 | 0.5% | 0.5% | 0.5% | 0.5% |
| Vanilla bean | 0.1% | 0.1% | 0.1% | 0.1% |
| extract, spray- | | | | |
| dried, Symrise | | | | |
| Compound 2, | — | — | 0.01% | 0.005% |
| Example 1 | | | | |
| D-tagatose | — | — | — | 0.1% |
| Milk 1.5% fat | ad 100% | ad 100% | ad 100% | ad 100% |
| content | | | | |

The solids were introduced and stirred up with the milk. The mixture was heated to 95° C. for 2 minutes with thorough stirring, decanted and cooled to 5-8° C.

In the case of preparation C, when tasted by testers, the sweetness of comparative preparation A containing 7.8% sucrose could be achieved (with a slightly delayed sweet sensation). Preparation C had a substantially much sweeter taste compared to comparative preparation B. Preparation D was comparable with C, but had an improved initial sweetness.

### Application Example 12

### Low-Fat Yogurts

A: comparative preparation with 10% sucrose
B, C: preparations according to the invention with sweetener mixture and 3′,7-dihydroxy-4′-methoxyflavan (compound 2, Example 1)

| | Preparation (amounts as % by weight) | | |
|---|---|---|---|
| Ingredient | A | B | C |
| Sucrose | 10% | 8% | 6% |
| Tagatose | — | — | 0.5% |
| Fructose | — | — | 0.5% |
| (Compound 2, | — | 0.05% | 0.025% |
| Example 1 | | | |
| Hesperetin | — | — | 0.005% |
| Yogurt, | ad 100% | ad 100% | ad 100% |
| 0.1% fat | | | |

The ingredients were mixed and cooled at 5° C.

### Application Example 13

### Use Together with Sweeteners in Low-Fat Yogurts

A: comparative preparation with sweetener mixture
B-D: preparations according to the invention with sweetener mixture and 3′,7-dihydroxy-4′-methoxyflavan (compound 2, Example 1)

| | Preparation (amounts in % by weight) | | | |
|---|---|---|---|---|
| Ingredient | A | B | C | D |
| D-tagatose | 0.482% | 0.482% | 0.482% | — |
| Sucralose | 0.003% | 0.003% | 0.003% | — |
| Aspartame | 0.005% | 0.005% | 0.005% | — |
| Acesulfame K | 0.01% | 0.01% | 0.01% | — |
| Rebaudioside | — | — | — | 0.050% |
| A 98% | | | | |
| Compound 2, | — | 0.05% | 0.025% | 0.025% |
| Example 1 | | | | |
| Hesperetin | — | — | 0.015% | 0.015% |
| Phloretin | — | — | 0.005% | 0.005% |
| Yogurt, | ad 100% | ad 100% | ad 100% | ad 100% |
| 0.1% fat | | | | |

The ingredients were mixed and cooled at 5° C.

### Application Example 14

### Mixed Milk Drinks

A, B: comparative preparations with sugar
C, D: preparations according to the invention with sugar and 3′,7-dihydroxy-4′-methoxyflavan (compound 2, Example 1)

Preparation (amounts in % by weight)

| Ingredient | A | B | C | D |
|---|---|---|---|---|
| Sucrose | 10.0 | 8.0 | 8.0 | 7.0 |
| Fructose | — | — | — | 0.5 |
| Compound 2, | — | — | 0.05 | 0.025 |
| Example 1 | | | | |
| Hesperetin | — | — | — | 0.025% |
| Phloretin | — | — | — | 0.005% |

| | |
|---|---|
| UHT-milk, | ad 100% |
| 1.5% fat | |

The ingredients were mixed, made up with milk, stirred thoroughly, poured into bottles and stored cooled at 5° C.

### Application Example 15

### Reduced-Sugar Tomato Ketchup

A: comparative preparation with sugar
B: comparative preparation with reduced sugar content (compared to A)
C-H: preparations according to the invention with reduced sugar content (compared to A) and 3′,7-dihydroxy-4′-methoxyflavan (compound 2, Example 1)

| | Preparation (amounts in % by weight) | | | | | | | |
|---|---|---|---|---|---|---|---|---|
| Ingredient | A | B | C | D | E | F | G | H |
| Common salt | 2 | 2 | 2 | 2 | 2 | 2 | 2 | 2 |
| Starch, Farinex | 1 | 1 | 1 | 1 | 1 | 1 | 1 | 1 |
| WM 55 | | | | | | | | |
| Sucrose | 12 | 9.6 | 9.2 | 8.4 | 9.6 | 9.6 | 8.4 | 8.4 |
| Tomato | 40 | 40 | 40 | 40 | 30 | 30 | 30 | 30 |
| concentrate × 2 | | | | | | | | |
| Glucose syrup 80 | 18 | 18 | 18 | 18 | 18 | 18 | 18 | 18 |
| Brix | | | | | | | | |
| Spirit vinegar | 7 | 7 | 7 | 7 | 3 | 3 | 3 | 3 |
| 10% | | | | | | | | |
| Compound 2, | | | 0.2 | 0.1 | 0.1 | 0.2 | 0.1 | 0.1 |
| Example 1, | | | | | | | | |
| 2.5% in | | | | | | | | |
| 1,2-propylene | | | | | | | | |
| glycol | | | | | | | | |
| Hesperetin 2.5% | | | | | 0.1 | | 0.2 | |
| in 1,2-propylene | | | | | | | | |
| glycol | | | | | | | | |
| Phloretin 2.5% | | | | 0.2 | 0.2 | | | 0.2 |
| in 1,2-propylene | | | | | | | | |
| glycol | | | | | | | | |

| | |
|---|---|
| Water | Make up to 100% |

The ingredients are mixed in the stated sequence and the finished ketchup is homogenized using an agitator, poured into bottles and sterilized.

### Application Example 16

### Reduced-Sugar Ice Cream

A: comparative preparation with sugar
B: comparative preparation with reduced sugar content (compared to A)
C-F: preparations according to the invention with reduced sugar content (compared to A) and hesperetin

| | Preparation (content in % by weight) | | | | | |
|---|---|---|---|---|---|---|
| Ingredient | A | B | C | D | E | F |
| Vegetable fat | 20.00 | 20.00 | 20.00 | 20.00 | 20.00 | 20.00 |
| melting range 35-40° C. | | | | | | |
| Sugar (Saccharose) | 12.00 | 8.00 | 8.00 | 8.00 | 8.00 | 8.00 |
| Skimmed milk powder | 5.00 | 5.00 | 5.00 | 5.00 | 5.00 | 5.00 |
| Glucose syrup 72% dry matter | 5.00 | 5.00 | 5.00 | 5.00 | 5.00 | 5.00 |
| Emulsifier SE 30 (Grindstedt | 0.65 | 0.65 | 0.65 | 0.65 | 0.65 | 0.65 |
| Products, Denmark) | | | | | | |
| Flavoring containing 0.1% | 0.20 | 0.20 | 0.20 | 0.20 | 0.20 | 0.20 |
| diacetyl and 1% vanillin | | | | | | |
| Compound 2, Example 1, | | | 0.20 | 0.10 | 0.20 | 0.10 |
| 2.5% in 1,2-propylene glycol | | | | | | |
| Hesperetin 2.5% in | | | | 0.10 | | 0.10 |
| 1,2-propylene glycol | | | | | | |
| Phloretin 2.5% in | | | | | 0.05 | 0.05 |
| ,2-propylene glycol | | | | | | |

| | |
|---|---|
| Skimmed milk | Make up to 100% |

The vegetable fat was heated to 58° C. Skimmed milk and glucose syrup were heated to 55° C. and sugar, skimmed milk powder and emulsifier and flavoring were added and the mixture was introduced into the vegetable fat. The mixture was homogenized using a through-flow high-pressure homogenizer (180/50 bar). The resulting mass was tempered for 1 minute at 78° C., then cooled to 2-4° C. and incubated at this temperature for 10 hours for maturing. The matured mass was then filled into containers and stored frozen at −18° C.

### Application Example 17

### Ice Cream Suitable for Diabetics

An ice cream suitable for diabetics was prepared from the following ingredients and filled into 95 ml portion tubs.

Concentrated, skimmed milk, fructose syrup, strawberry pieces and strawberry puree (15%), vegetable fat, diet chocolate chips (3.5% with soya lecithin emulsifier), whey product, beetroot juice, locust bean gum, guar gum, carrageen, emulsifier (E 471), gelatin, acidifying agent citric acid, strawberry flavoring (containing 1% by weight 3′,7-dihydroxy-4′-methoxyflavan (compound 2, Example 1), based on the total weight of the strawberry flavoring), carotene coloring.

Nutritional value (per 95 ml):

Protein 1.8 g, carbohydrates 13.3 g (of which fructose 9.5 g), fat 4.2 g.

### Application Example 18

### Diet Chocolate Based on Maltitol

A chocolate suitable for diabetics was prepared from the following ingredients and poured into rectangular bars:

Maltitol, hazelnut mass, cocoa butter, skimmed milk powder, cocoa mass, inulin, concentrated butter, emulsifier soya lecithins, vanilla flavoring (containing vanilla bean extract), vanillin and 1% by weight 3′,7-dihydroxy-4′-methoxyflavan (compound 2, Example 1), based on the total weight of the vanilla flavoring.

Nutritional value (per 100 g): protein 8 g, carbohydrates 43 g (of which maltitol 34 g), fat 34 g.

### Application Example 19

### Diet Chocolate Based on Fructose

A chocolate suitable for diabetics was prepared from the following ingredients and poured into rectangular bars:

Cocoa mass, fructose, skimmed milk powder, cocoa butter, inulin, concentrated butter, emulsifier soya lecithin, walnuts, cooking salt, vanilla flavoring (containing vanillin and 1% by weight 3′,7-dihydroxy-4′-methoxyflavan (compound 2, Example 1), based on the total weight of the vanilla flavoring).

Nutritional value (per 100 g):

Protein 8.8 g, carbohydrates 34 g (of which fructose 23 g, lactose 7.5 g, saccharose 1.4 g), fat 36 g; dietary fiber 18.5 (of which 12.2 g inulin); sodium: 0.10 g. Cocoa content at least 50% by weight.

### Application Example 20

### Reduced-Sugar Muesli Mixture

| | | A (% by weight) | B (% by weight), |
|---|---|---|---|
| | | Comparative | According to the |
| No. | Ingredient | preparation | invention |
| 1 | Oat flakes | 17.00 | 18.90 |
| 2 | Crunchy oat flake clusters | 10.00 | 12.00 |
| 3 | Rice Crispies | 16.90 | 17.80 |
| 4 | Cornflakes | 16.50 | 17.50 |
| 5 | Currants | 3.50 | 3.50 |
| 6 | Hazelnuts, chopped | 2.50 | 2.50 |
| 7 | Glucose syrup from wheat, | 9.50 | 9.50 |
| | DE 30 | | |
| 8 | Saccharose | 20.00 | 14.00 |
| 9 | Water | 4.00 | 4.00 |
| 10 | Citric acid powder, | 0.10 | 0.10 |
| | anhydrous | | |
| 11 | Flavoring, containing 2.5% | — | 0.20 |
| | by weight of 3′,7- | | |
| | dihydroxy-4′-methoxy- | | |
| | flavan (compound 2, | | |
| | Example 1), based on the | | |
| | total weight of the flavoring | | |

Ingredients Nos. 1 to 6 are mixed in each case in a rotary drum (Mix 1). Ingredients Nos. 7 to 9 are heated and ingredient No. 10 (in recipe B also ingredient No. 11) is added (Mix 2). Mix 2 is added to Mix 1 and then they are thoroughly mixed together. Finally, the resulting muesli mixture is turned out onto a baking tray and dried in an oven for 8 minutes at 130° C.

### Application Example 21

### Reduced-Sugar Fruit Gums

| | A (% by weight), | B (% by weight), |
|---|---|---|
| | Comparative | According to the |
| Ingredient | preparation | invention |
| Water | 23.70 | 25.70 |
| Saccharose | 34.50 | 8.20 |
| Glucose syrup, DE 40 | 31.89 | 30.09 |
| Iso Syrup C* Tru Sweet 01750 | 1.50 | 2.10 |
| (Cerestar GmbH) | | |
| Gelatine 240 Bloom | 8.20 | 9.40 |
| Polydextrose (Litesse ® Ultra, | — | 24.40 |
| Danisco Cultor GmbH) | | |
| Yellow and red colorings | 0.01 | 0.01 |
| Citric acid | 0.20 | |
| Cherry flavoring, containing | — | 0.10 |
| 2.5% by weight of 3′,7- | | |
| dihydroxy-4′-methoxyflavan | | |
| (compound 2, Example 1) | | |
| based on the flavoring | | |

Note:
polydextrose is itself a non-sweet-tasting polysaccharide with a low calorific value.

### Application Example 22

### Choco-Cappuccino Ice Cream

| | A (% by weight), | B (% by weight), |
|---|---|---|
| | Comparative | According to the |
| Ingredient | preparation | invention |
| Glucose-fructose syrup | 14.10 | 14.10 |
| Saccharose | 10.00 | 7.50 |
| Skimmed milk powder | 5.00 | 5.00 |
| Cream (36% fat content) | 24.00 | 24.00 |
| Emulsifier and stabilizer | 0.50 | 0.50 |
| Cremodan ® 709VEG (Danisco) | | |
| Cocoa powder | 5.975 | 5.975 |
| Carrageenan | 0.025 | 0.025 |
| Water | 40.20 | 42.50 |
| Cappuccino flavoring | 0.20 | 0.20 |
| 3′,7-dihydroxy-4′- | — | 0.20 |
| methoxyflavan (compound 2, | | |
| Example 1) 2.5% in 1,2-propylene | | |
| glycol/ethanol | | |

### Application Example 23

### Gelatin Capsules for Direct Consumption

| | A (% by | B (% by | C (% by |
|---|---|---|---|
| Ingredient | weight) | weight) | weight) |
| Gelatin sheath: | | | |
| Glycerin | 2.014 | 2.014 | 2.014 |
| Gelatin 240 Bloom | 7.91 | 7.91 | 7.91 |
| Sucralose | 0.065 | 0.065 | 0.065 |
| Allura Red | 0.006 | 0.006 | 0.006 |
| Brilliant Blue | 0.005 | 0.005 | 0.005 |
| Core composition: | | | |
| Vegetable oil-triglyceride | 79.49 | 68.55 | 58.55 |
| (coconut oil fraction) | | | |
| Orange flavoring, containing | 10.0 | 20.0 | 28.65 |
| 1% by weight dihydroxy-4′- | | | |
| methoxyflavan (compound 2, | | | |
| Example 1), based on the total | | | |
| weight of the flavoring | | | |
| Rebaudioside A 98% | 0.05 | 0.05 | — |
| 2-Hydroxypropylmenthylcarbonate | 0.33 | 0.20 | — |
| 2-Hydroxyethylmenthylcarbonate | — | 0.20 | 1.00 |
| (1R,3R,4S) Menthyl-3-carboxylic | — | 0.55 | 0.50 |
| acid-N-ethylamide (WS-3) | | | |
| (−)-Menthone glycerin acetal | — | 0.30 | 0.80 |
| (Frescolat MGA) | | | |
| Vanillin | 0.07 | — | 0.10 |

The gelatin capsules suitable for direct consumption were prepared according to WO 2004/050069 and had a diameter of 5 mm and the weight ratio of core material to sheath material was 90:10. The capsules opened in the mouth in less than 10 seconds and dissolved completely in less than 50 seconds.

1. A method of using
a compound of formula (I), 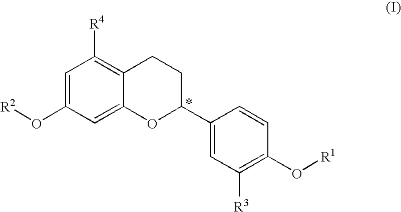
or
a salt of a compound of formula (I)
or
a mixture of two or more different compounds of formula (I), two or more different salts of compounds of formula (I) or one or more different compounds of formula (I) and one or more different salts of compounds of formula (I),
wherein:
R¹ and R² independently of one another represent hydrogen, methyl or ethyl
R³ and R⁴ independently of one another represent hydrogen, hydroxyl, methoxy or ethoxy; and
the configuration at the chiral carbon atom is (R) or (S),
to synergistically enhance a sweet taste of a sweet-tasting substance.

2. The method according to claim 1 comprising using at least one compound of formula (I) is selected from the group consisting of:
4′,7-dihydroxyflavan (compound 1),
3′,7-dihydroxy-4″-methoxyflavan (compound 2),
4′,7-dihydroxy-3″-methoxyflavan (compound 3),
4′-hydroxy-7-methoxyflavan (compound 4),
4′,7-dimethoxy-3′-hydroxyflavan (compound 5),
3′,7-dimethoxy-4′-hydroxyflavan (compound 6),
3′,4′-dihydroxy-7-methoxyflavan (compound 7),
5,7-dimethoxy-4′-hydroxyflavan (compound 8),
4′-hydroxy-3′,5,7-trimethoxyflavan (compound 9),
3′,4′-dihydroxy-5,7-dimethoxyflavan (compound 10),
4′,5-dihydroxy-3′,7-dimethoxyflavan (compound 11),
3′,4′,5,7-tetrahydroxyflavan (compound 12),
3′,4′,5-trihydroxy-7-methoxyflavan (compound 13),
4′,5,7-trihydroxyflavan (compound 14),
3′,5,7-trihydroxy-4′-methoxyflavan (compound 15), and
4′,5,7-trihydroxy-3′-methoxyflavan (compound 16),
and wherein the configuration at the chiral carbon atom of the compound of formula (I) is (R) or (S).

3. The method according to claim 1, wherein at least one compound of formula (I) has 1-3 hydroxyl groups.

4. The method according to claim 1, wherein at least one of R² and R⁴ is hydrogen.

5. The method according to claim 4, wherein
R¹ represents hydrogen or methyl;
and/or
R³ represents hydrogen or hydroxyl.

6. The method according to claim 1, wherein at least one compound of formula (I) is selected from the group consisting of 4′,7-dihydroxyflavan (compound 1) and 3′,7-dihydroxy-4′-methoxyflavan (compound 2), and wherein the configuration at the chiral carbon atom is (R) or (S).

7. The method of synergistically enhancing the sweet taste of a the sweet-tasting substance of claim 1 using the mixture of two different compounds of formula (I),
wherein
R¹, R², R³, and R⁴ of the two compounds are identical; and
the configuration at the chiral carbon atom of one compound is (R) and the configuration at the chiral carbon atom of the other compound is (S).

8. The method according to claim 7, wherein the mixture of two different compounds of formula (I) is a racemic mixture.

9. The method of synergistically enhancing a sweet taste of a sweet-tasting substance comprising using at least one compound of formula (I), according to claim 1, that is (S)-3′,7-dihydroxy-4′-methoxyflavan (compound S-2).

10. The method according to claim 9, wherein the mixture of two or more different compounds of formula (I) comprises either no (R)-3′,7-dihydroxy-4′-methoxyflavan or comprises (R)-3′,7-dihydroxy-4′-methoxyflavan in a quantity which is less than the quantity of (S)-3′,7-dihydroxy-4′-methoxyflavan (compound S-2) in said mixture.

11. A method of using a salt or a mixture of
two or more different salts of compounds of formula (I),
or
one or more different compounds of formula (I), and one or more different salts of compounds of formula (I)
according to claim 1,
wherein the counter cation(s) of the salt(s) of the compounds of formula (I) is/are selected from the group consisting of Na⁺, K⁺, NH₄⁺, Ca²⁺ and Zn²⁺.

12. The method of using at least one compound of formula (I) according to claim 6 in a mixture comprising
a sweet-tasting substance (A) and an unpleasant-tasting substance (B)
or
a both sweet and unpleasant-tasting substance (C),
(a) for synergistically enhancing a sweet taste of the sweet-tasting substance (A) or (C)
and
(b) for masking or reducing an unpleasant taste sensation of the unpleasant-tasting substance (B) or (C).

13. The method according to claim 12, wherein the unpleasant taste sensation of the unpleasant-tasting substance (B) or (C) is a bitter taste sensation.

14. The method according to claim 13, wherein
the bitter-tasting substance (B) or
the both sweet and bitter-tasting substance (C)
is a stevioglycoside selected from the group consisting of rebaudioside A, rubusoside, dulcoside, mogroside, phyllodulcin, glycyrrhetinic acid or extracts of Stevia spp., luo han guo, Rubus suavissimus, Hydrangea dulcis and glycyrrhyza glabra.

15. A method of using a plant extract comprising:
the compound of formula (I)
or
the salt of a compound of formula (I)
or
the mixture of two or more different compounds of formula (I), two or more different salts of compounds of formula (I) or one or more different compounds of formula (I) and one or more different salts of compounds of formula (I),
according to claim 1 to synergistically enhance the sweet taste of a sweet-tasting substance.

16. The method of using a plant extract according to claim 15 in a mixture comprising:
a sweet-tasting substance (A) and a bitter-tasting substance (B)
or
a both sweet and bitter-tasting substance (C)
(a) to synergistically enhance a sweet taste of the sweet-tasting substance (A) or (C); and
(b) to mask or reduce a bitter taste sensation of the bitter-tasting substance (B) or (C).

17. The method of using a plant extract according to claim 16, wherein
the bitter-tasting substance (B) or
the both sweet and bitter-tasting substance (C)
is a stevioglycoside selected from the group consisting of rebaudioside A, rubusoside, dulcoside, mogroside, phyllodulcin, glycyrrhetinic acid or extracts of Stevia spp., luo han guo, Rubus suavissimus, Hydrangea dulcis and glycyrrhyza glabra.

18. A method of making a pharmaceutical preparation intended for oral ingestion, a cosmetic preparation or a preparation used for nutrition, oral care or enjoyment comprising the method according to claim 1.

19. A method of flavoring a composition for synergistically enhancing a sweet taste of a sweet-tasting substance (A) and for masking or reducing a bitter taste sensation of a bitter-tasting substance (B), and for masking or reducing a bitter taste sensation of a both sweet and bitter-tasting substance (C), comprising using a composition comprising:
(i)—a compound of formula (I)
or
a salt of a compound of formula (I)
or
a mixture of two or more different compounds of formula (I), two or more different salts of compounds of formula (I) or one or more different compounds of formula (I) and one or more different salts of compounds of formula (I),
according to claim 1
and
(ii) at least one flavoring or taste-imparting substance selected from the group consisting of vanillin, ethyl vanillin, ethylvanillinisobutyrate, 2,5-dimethyl-4-hydroxy-3(2H)-furanone and derivatives, homofuronol, maltol and derivatives, coumarin and derivatives, gamma-lactones, delta-lactones, methylsorbate, divanillin, 4-hydroxy-2-ethyl-5-methyl-3(2H)furanone, 4-hydroxy-5-ethyl-2-methyl-3(2H)furanone, 2-hydroxy-3-methyl-2-cyclopentenones, 3-hydroxy-4,5-dimethyl-2(5H)-furanone, fruit esters and fruit lactones 4-(p-hydroxyphenyl)-2-butanone, 1,1-dimethoxy-2,2,5-trimethyl-4-hexane, 2,6-dimethyl-5-hepten-1-al and phenylacetaldehyde,
and/or
(iii) at least one
substances for masking or reducing a bitter taste sensation selected from the group consisting of sodium salts, homoeriodictyol or the sodium salts thereof, 2,4-dihydroxybenozoic acid vanillylamide, gamma-amino butyric acid, pellitorine and gingerdiones
and/or
at least one substance for enhancing a sweet taste sensation, selected from the group consisting of hesperetin, hydroxyphenylalkadiones, deoxybenzoins, 4-hydroxychalcones, propenylphenylglycosides and divanillins.

20. The method of flavoring a composition comprising using a composition according to claim 19 in a mixture comprising
a sweet-tasting substance (A) and/or a bitter-tasting substance (B)
or
a both sweet and bitter-tasting substance (C)
(a) to synergistically enhance a sweet taste of the sweet-tasting substance (A) or (C) and
(b) to mask or reduce an unpleasant taste sensation of the unpleasant-tasting substance (B) or (C).

21. The method according to claim 20, wherein
the bitter-tasting substance (B) or
the both sweet and bitter-tasting substance (C)
is a stevioglycoside selected from the group consisting of rebaudioside A, rubusoside, dulcoside, mogroside, phyllodulcin, glycyrrhetinic acid or extracts of Stevia spp., luo han guo, Rubus suavissimus, Hydrangea dulcis and glycyrrhyza glabra.

22. A preparation comprising the ingredients
(I)—the compound of formula (I)
or
the salt of a compound of formula (I)
or
the mixture of two or more different compounds of formula (I), two or more different salts of compounds of formula (I) or one or more different compounds of formula (I) and one or more different salts of compounds of formula (I),
according to claim 1 and
(II) one or more sweet-tasting substances (A) and/or both sweet and bitter-tasting substances (C), wherein the substances(s) (A) or (C) is/are not a compound of formula (I) or a salt thereof,
and the total amount of compounds of formula (I) or salts of the compound of formula (I) in the preparation is sufficient for synergistically enhancing a sweet taste sensation of the substance(s) (A) or (C).

23. The preparation according to claim 22, wherein the preparation is a preparation used for nutrition, oral care or enjoyment or a cosmetic preparation or a pharmaceutical preparation intended for oral ingestion.

24. The preparation according to claim 23, wherein the preparation contains 0.0001% by weight (1 ppm) to 0.5% by weight (5000 ppm) of ingredient (I), based on the total weight of the preparation.

25. The preparation according to claim 22, wherein the preparation is a semi-finished product suitable for the production of a preparation used for oral care or enjoyment or a cosmetic preparation or a pharmaceutical preparation intended for oral ingestion.

26. The preparation according to claim 25, wherein the semi-finished product contains 0.0001% by weight to 95% by weight of ingredient (I), based on the total weight of the semi-finished product.

27. The preparation according to claim 22, wherein the total amount of compounds of formula (I) or salts of the compound of formula (I) in the preparation is sufficient to impart the same or an enhanced sweetness sensation when compared to a preparation which, with an otherwise identical composition, does not contain a compound of formula (I) or salt of the compound of formula (I) but does contain at least 1.05 times the amount of sweet-tasting substance(s) (A) or (C).

28. The preparation according to claim 22, wherein ingredient (I) comprises
a compound of formula (I) as described in claim 6
or
a mixture as described in claim 7.

29. A preparation according to claim 28, wherein ingredient (II) comprises
one or more both sweet and bitter-tasting substances (C)
and/or
one or more bitter-tasting substances (B), wherein (B) is not a compound or salt of formula (I),
and the total amount of compounds of formula (I) or salts of the compound of formula (I) in the preparation is sufficient to mask or reduce a bitter taste sensation of the substance(s) (B) or (C).

30. A preparation according to claim 29, wherein at least one of the one or more both sweet and bitter-tasting substances (C) is a stevioglycoside selected from the group consisting of rebaudioside A, rubusoside, dulcoside, mogroside, phyllodulcin, glycyrrhetinic acid or extracts of Stevia spp., luo han guo, Rubus suavissimus, Hydrangea dulcis and glycyrrhyza glabra.

31. A preparation according to claim 30, wherein the total amount of bitter-tasting substances (B) and/or both sweet and bitter-tasting substances (C) in the preparation is sufficient to be detected as a bitter taste in a comparative preparation which, with an otherwise identical composition, does not contain a compound of formula (I) or a salt of the compound of formula (I), and the total amount of compounds of formula (I) or salts of the compound of formula (I) in the preparation is sufficient for masking or, compared with the comparative preparation, for reducing the bitter taste of the substance(s) (B) or (C).

32. A preparation according to claim 31 further comprising at least one substance for masking or reducing a bitter, metallic, chalky, acidic or astringent taste sensation or for enhancing a sweet, salty or umami taste sensation.

33. A preparation according to claim 32, wherein
ingredient (I) is present in the form of a plant extract and/or
ingredient (II) is present in the form of an extract of Stevia rabaudiana.

34. A process for synergistically enhancing a sweet taste of a sweet-tasting substance, comprising the steps:
i) preparing a sweet-tasting substance (A) or of a both sweet and bitter-tasting substance (C), and
ii) preparing
a compound of formula (I),
or
a salt of a compound of formula (I)
or
a mixture of two or more different compounds of formula (I), two or more different salts of compounds of formula (I) or one or more different compounds of formula (I) and one or more different salts of compounds of formula (I),
according to claim 1;
and
iii) blending the components prepared in steps i) and ii) in a ratio to one another such that the sweet taste of substance (A) or (C) is synergistically enhanced.

35. The process according to claim 34, wherein the process is used
(a) for synergistically enhancing the sweet taste
and
(b) for masking or reducing a bitter taste sensation
of a both sweet and bitter-tasting substance (C), comprising the steps:
i) preparing a both sweet and bitter-tasting substance (C),
ii) preparing
a compound of formula (I),
or
a salt of a compound of formula (I)
or
a mixture of two or more different compounds of formula (I), two or more different salts of compounds of formula (I) or one or more different compounds of formula (I) and one or more different salts of compounds of formula (I),
according to claim 1;
and
iii) blending the components prepared in steps i) and ii) in a ratio to one another such that (a) a sweet taste of the both sweet and bitter-tasting substance (C) is synergistically enhanced and (b) a bitter taste sensation of the both sweet and bitter-tasting substance (C) is masked or reduced.
